(19) United States
(12) Patent Application Publication
(10) Pub. No.: US 2026/0000864 A1
(43) Pub. Date: Jan. 1, 2026

(54) INSERT CATHETER FOR ROBOTIC MULTI-CATHETER ASSEMBLY

(71) Applicant: Imperative Care, Inc., Campbell, CA (US)

(72) Inventors: Theodore August Jourdan, Mt. Pleasant, SC (US); Craig Mar, Fremont, CA (US); Steven Thomas Meyer, Oakland, CA (US); Ketan Pranlal Muni, San Jose, CA (US); Avila Teresa Monica Salathuraj, Fremont, CA (US)

(21) Appl. No.: 19/249,152

(22) Filed: Jun. 25, 2025

Related U.S. Application Data
(60) Provisional application No. 63/664,547, filed on Jun. 26, 2024.

(51) Int. Cl. A61M 25/00

(52) U.S. Cl. CPC A61M 25/0054; A61M 25/0052

(57) ABSTRACT

A neurovascular catheter for delivery through an aorta and into an ostium can include a tubular body between a proximal end to a distal end. The tubular body can have a length of 140 cm to 180 cm, a 5 Fr size, and an internal diameter of about 0.065″ to about 0.067″. The tubular body can include a proximal portion and a distal portion. The distal portion can include a polymer layer and a layer of braids. The proximal portion can include a hypotube and the layer of braids.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the priority benefit under 35 U.S.C. § 119(e) to U.S. Provisional Application No. 63/664,547, filed Jun. 26, 2024, hereby incorporated by reference herein in its entirety.

### RELATED APPLICATIONS

US2024/0382668 (the '668 publication), published on Nov. 21, 2024, describes various example embodiments and features related to apparatuses, systems, and methods for fluidics management and delivery and specifically relating to fluidics control for multiple catheter stacks during medical procedures, either manual or robotically driven. The '668 publication is expressly incorporated by reference in its entirety and is part of this disclosure. The embodiments described below are compatible with and can be part of the embodiments described in the '668 publication, and some or all of the features described below can be used or otherwise combined together or with any of the features described in the '668 publication.

US2024/0197418 (the '418 publication), published on Jun. 20, 2024, describes various example embodiments and features related to apparatuses, systems, and methods with removable hubs for manual and robotic procedures. The '418 publication is expressly bodily incorporated in its entirety and is part of this disclosure. The embodiments described below are compatible with and can be part of the embodiments described in the '418 publication, and some or all of the features described below can be used or otherwise combined together or with any of the features described in the '418 publication. For example, certain embodiments described below can be used in manual and robotic procedures as described in the '418 publication.

### BACKGROUND

### Field

The present application relates to neurovascular procedures, and more particularly, to certain catheters in catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neurointerventions. Neurointerventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovascular is challenging to achieve, especially Type III arches. For example, during current neurothrombectomy procedures physicians must remove the guidewire from the catheter to perform dye injections. Many dye injections are needed to navigate through the complex vasculature up to the brain, so guidewires must be removed over 10 times a procedure. The extra step of removing the guidewire increases the duration of the procedure increasing costs time and induces risk of introducing an air bubble to the patient. Even once supra-aortic access is achieved, adapting the system for neurovascular treatments remains time consuming, and requires guidewire and insert catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

In some aspects, the techniques described herein relate to a neurovascular catheter for delivery through an aorta and into an ostium, the catheter including: a tubular body extending between a proximal end to a distal end, wherein the tubular body can have a length of about 140 cm to about 180 cm, a 5 Fr size, and an internal diameter of about 0.065″ to about 0.067″, the tubular body including a proximal portion and a distal portion, wherein the distal portion can include a polymer layer, and wherein the proximal portion can include a hypotube, wherein the tubular body can include a layer of braids extending at least partially along the proximal portion and at least partially along the distal portion.

In some aspects, the layer of braid can extend from the proximal end up to a predetermined distance from the distal end.

In some aspects, the predetermined distance can be about 1 cm to about 2 cm.

In some aspects, a distal most portion of the catheter within the predetermined distance from the distal end can include a polymer with a lowest hardness value.

In some aspects, a distal most portion of the catheter within the predetermined distance from the distal end can include a radiopaque marker.

In some aspects, the tubular body can include a pre-shaped tip, the pre-shaped tip including one or more curves.

In some aspects, the distal portion of the tubular body can include a segment with Tungsten additives, the segment extending over the pre-shaped tip.

In some aspects, a distal portion of the layer of braids can be annealed.

In some aspects, the distal portion that is annealed can include a soft tip.

In some aspects, the distal portion that is annealed can further include a section with a radiopaque marker.

In some aspects, the distal portion that is annealed can be shorter than a length of the pre-shaped tip.

In some aspects, a length of the distal portion can depend on a shape of the pre-shaped tip.

In some aspects, the length of the distal portion of the layer of braids can be the shortest for the pre-shaped tip with a single curve.

In some aspects, the lair of braids can be located radially inward of the polymer layer.

In some aspects, the layer of braids can be located radially outward of the hypotube.

In some aspects, the hypotube can be made of Nitinol.

In some aspects, the hypotube can include a laser cut pattern.

In some aspects, the laser cut pattern can be a flexible laser cut pattern on proximal and distal ends of the hypotube.

In some aspects, the hypotube can be less stiff in proximal and distal ends with the flexible laser cut pattern.

In some aspects, the flexible laser cut pattern can comprise uninterrupted spiral cuts.

In some aspects, the laser cut pattern from a proximal end to a distal end of the hypotube can vary in one or more of different cut per rotation (CPR), uncut degree, pitch, and/or Kerf.

In some aspects, the hypotube can be configured to be packaged in a bent configuration and can further be configured to resume a straight configuration upon being unpackaged.

In some aspects, the distal portion of the tubular body can further include an outer polymer layer.

In some aspects, the polymer layer can overlap with the hypotube by a predetermined length.

In some aspects, the predetermined length can be about 1 mm.

In some aspects, the distal portion of the tubular body can be about 35 cm.

In some aspects, the tubular body can further include an inner lining layer extending from the proximal end to the distal end of the tubular body.

In some aspects, the length of the tubular body can be at least 175 cm, wherein the catheter can be configured to be driven by a robotic system.

In some aspects, the braid can include a 32-carrier design.

In some aspects, the braid can include a diamond pattern.

In some aspects, a stiffness of the hypotube can be greater than a stiffness of the polymer layer.

In some aspects, the stiffness of the polymer layer can increase from the distal end to the proximal end.

In some aspects, the techniques described herein relate to a method for delivering a fluid to a target site of a patient's vasculature using a robotic catheter system, the method comprising: moving a distal end of a guiding element towards the target site; moving a distal end of the catheter having any of the aspects described above while at least a portion of the guiding element is positioned in a lumen of the catheter; and providing contrast media into a proximal end of the catheter at a pressure of less than or equal to about 400 psi while at least a portion of the guiding element is positioned in the lumen of the catheter such that the provided contrast media propagates through the lumen along an exterior surface of the guiding element and out of the distal end of the catheter, wherein the lumen and the guiding element can be dimensioned to create an effective cross-sectional area greater than or equal to about 0.001257 square inches between an exterior surface of the guiding element and an interior surface of the catheter and provide a predetermined flow rate of the contrast media out of the distal end of the catheter.

In some aspects, the predetermined flow rate can be at least about 3 cc's per second.

In some aspects, the effective cross-sectional area can be annular shaped or eccentrically annular shaped.

In some aspects, the method can further include providing the contrast media while moving at least one of the guiding element or the catheter towards the target site.

In some aspects, the guiding element can be coupled to a first hub and the catheter can be coupled to a second hub, and wherein the first hub can be magnetically coupled to a first carriage of drive assembly through a sterile barrier, and the second hub can be magnetically coupled to a second carriage of the drive assembly through the sterile barrier, and moving the distal end of the guiding element and the distal end of the catheter can include moving the first carriage and moving the second carriage.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an insert catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 is a side elevational cross-section through a distal portion of a catheter such as any of those shown in FIGS. 5A and 5B.

FIG. 7 illustrates a side elevational schematic view of an interventional device assembly for supra-aortic access and neuro-interventional procedures.

FIG. 8 is a representation of an example of a distal end view of a concentric stack including a guide catheter, a procedure catheter, am insert catheter, and a guiding element (e.g., a guidewire).

FIG. 9A illustrates a cross sectional view of an example insert catheter.

FIG. 9B illustrates the example insert catheter of FIG. 9A.

FIG. 9C illustrates a distal portion of the catheter of FIG. 9B.

FIG. 9D illustrates a region of braid-to-braid hypotube transition.

FIG. 9E illustrates a partial cross-section of the catheter of FIG. 9B.

FIG. 10A is a plan view of an example insert catheter having a distal portion of a first shape.

FIG. 10B is an elevational view of the insert catheter of FIG. 10A.

FIG. 10C is a cross-sectional view of the insert catheter of FIG. 10A along axis A-A.

FIG. 10D is a detailed view of a distal end of the insert catheter of FIG. 10A.

FIG. 11A is a plan view of an example insert catheter having a distal portion of a second shape.

FIG. 11B is an elevational view of the insert catheter of FIG. 11A.

FIG. 11C is a cross-sectional view of the insert catheter of FIG. 11A along axis A-A.

FIG. 11D is a detailed view of a distal end of the insert catheter of FIG. 11A.

FIG. 12A is a plan view of an example insert catheter having a distal portion of a second shape.

FIG. 12B is an elevational view of the insert catheter of FIG. 12A.

FIG. 12C is a cross-sectional view of the insert catheter of FIG. 12A along axis A-A.

FIG. 12D is a detailed view of a distal end of the insert catheter of FIG. 12A.

FIG. 12E is a plan view of an example rapid exchange insert catheter having a distal portion of the second shape.

FIGS. 13A and 13B illustrate an example hypotube.

FIG. 13C illustrates an example flexible laser cut pattern.

FIG. 13D illustrates another example hypotube.

FIGS. 14A-14C illustrate an example hypotube.

FIG. 14D illustrates a catheter incorporating the hypotube of FIG. 14A.

FIG. 14E illustrates a hypotube transition portion of the catheter of FIG. 14D.

FIGS. 15A and 15B schematically illustrate a force sensor integrated into the sidewall of the catheter.

FIG. 16 schematically illustrates a dual encoder torque sensor for use with a catheter of the present disclosure.

FIGS. 17A-17E depict an example sequence of steps of introducing a catheter assembly configured to achieve supra-aortic access and neurovascular site access.

FIG. 18 illustrates a system diagram of an embodiment of a medical device operation system.

FIG. 19 illustrates a perspective view of an embodiment of a hub assembly.

FIG. 20 illustrates an embodiment of a drive table.

FIG. 21 illustrates an embodiment of a fluidics assembly.

FIG. 22 illustrates an embodiment of an embodiment of a fluidics assembly.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. In some embodiments, each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

### Example Interventional Setup

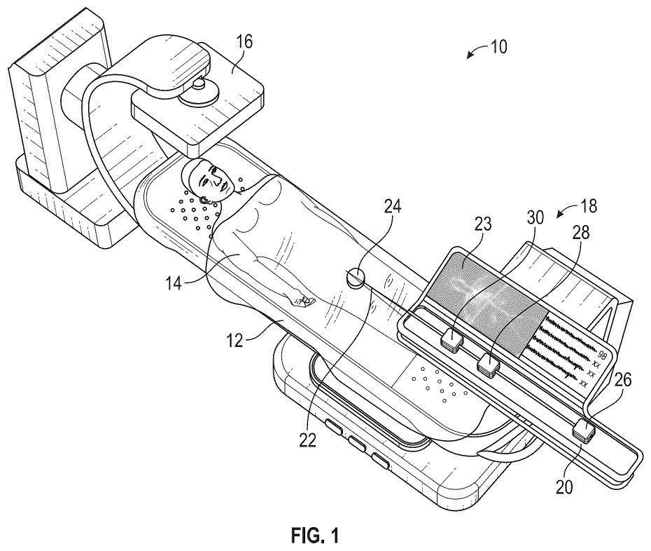

FIG. 1 is a schematic perspective view of an interventional setup 10 having a patient support table 12 for supporting a patient 14. An imaging system 16 may be provided, along with a robotic interventional device drive system 18 in accordance with the present disclosure.

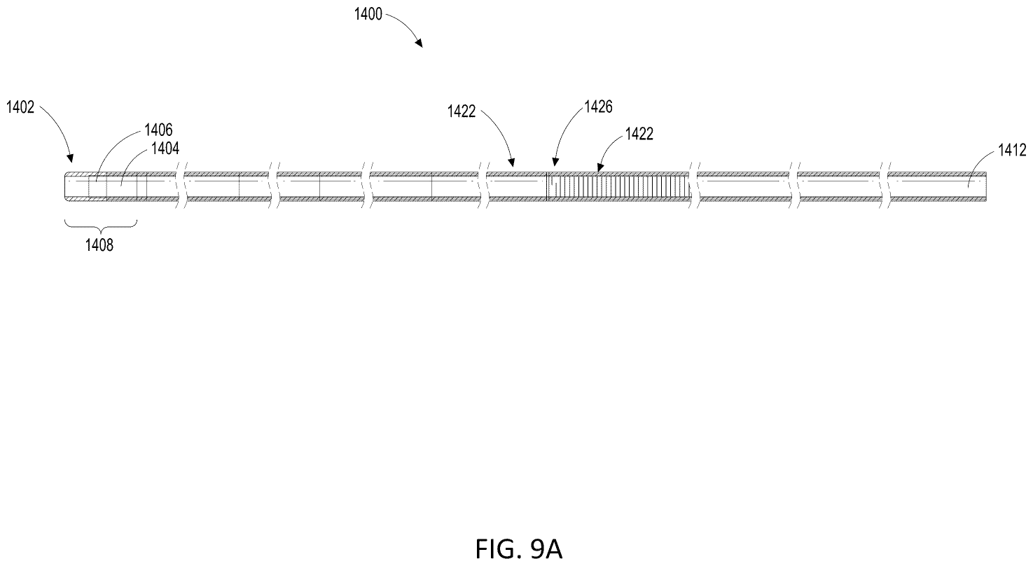

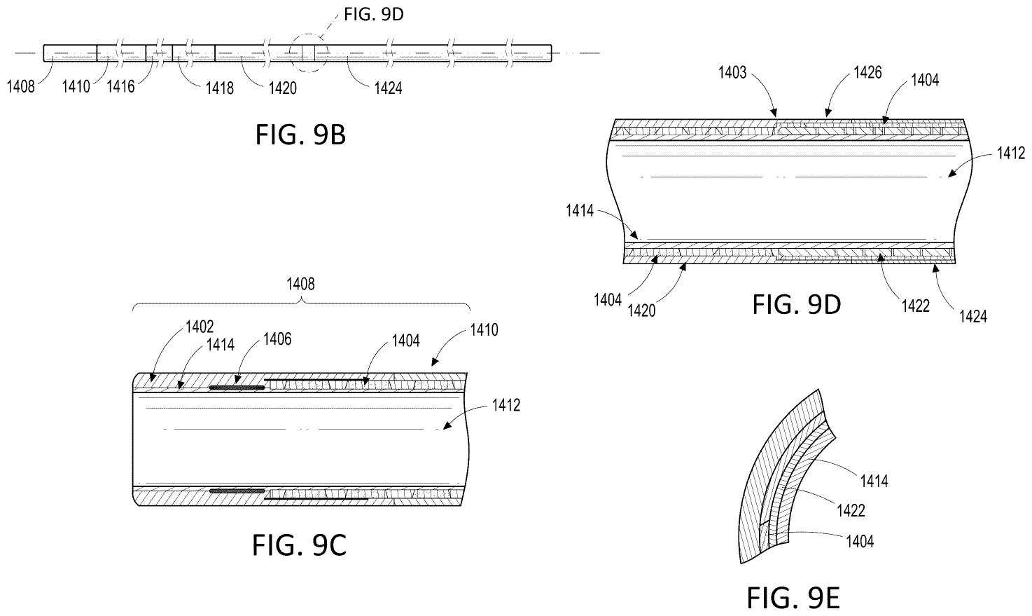

The drive system 18 may include a support table 20 for supporting, for example, a guidewire hub 26, an insert catheter hub 28 and a guide catheter hub 30. In the present context, the term “insert catheter” (also may be referred to as an “access catheter”) can be any catheter having a lumen with at least one distally facing or laterally facing distal opening, which may be utilized to aspirate thrombus, provide access for an additional device to be advanced therethrough or therealong, or to inject saline or contrast media or therapeutic agents. FIGS. 9A-14E illustrate certain examples of an insert catheter, which will be described in greater details below.

More or fewer interventional device hubs may be provided depending upon the desired clinical procedure. For example, in certain embodiments, a diagnostic angiogram procedure may be performed using only a guidewire hub 26 and an insert catheter hub 28 for driving a guidewire and an insert catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An insert catheter hub 28 is also carried by the support table 20 and is movable along the table to advance the insert catheter into and out of the patient 14. The insert catheter hub may also be configured to rotate the insert catheter in response to manipulation of a rotation control and may also be configured to laterally deflect a deflectable portion of the insert catheter, in response to manipulation of a deflection control.

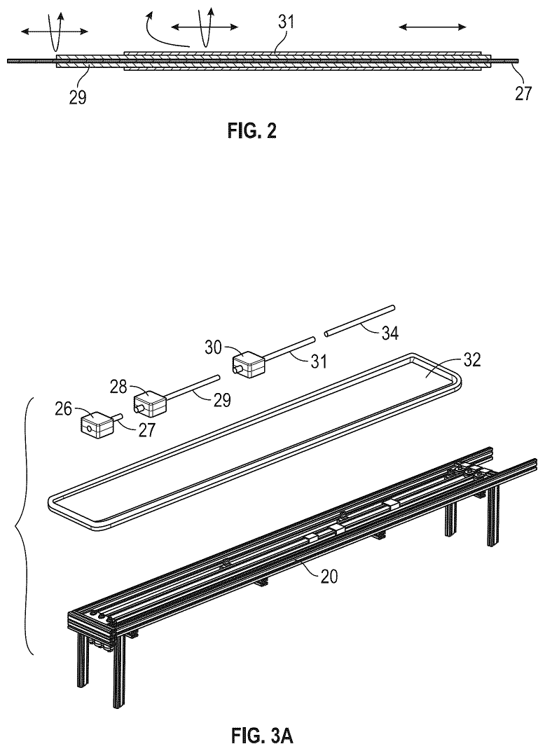

FIG. 2 is a longitudinal cross section schematically showing the motion relationship between a guidewire 27 having two degrees of freedom (axial and rotation), an insert catheter 29 having three degrees of freedom (axial, rotational and lateral deflection) and a guide catheter 31, having one degree of freedom (axial).

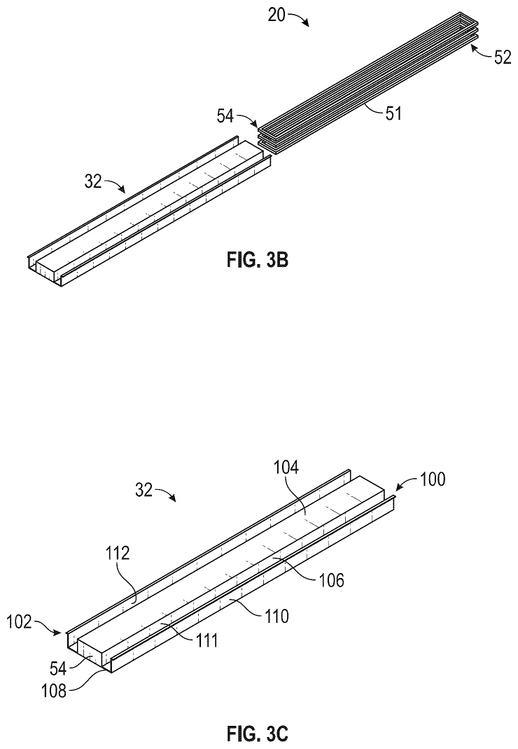

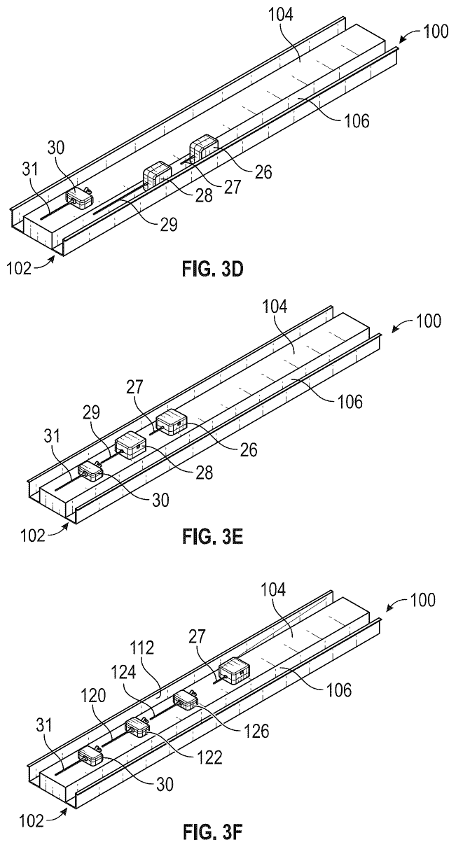

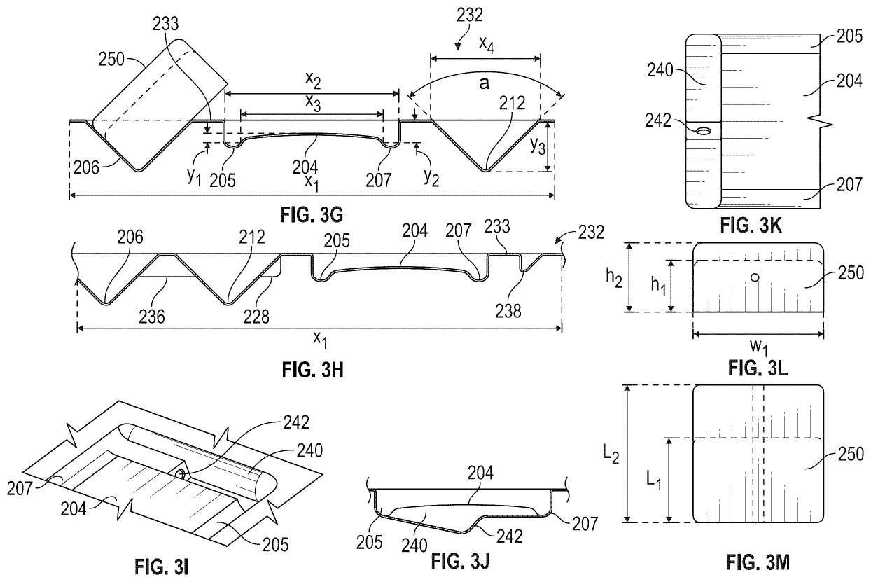

Referring to FIG. 3A, the support table 20 includes a drive mechanism described in greater detail below, to independently drive the guidewire hub 26, insert catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may include a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments, the hollow extending element is a set of telescoping portions that nest inside each other and enclose a shaft between the hubs. In some embodiments, the hollow extending element has a proximal (closest to insertion point) and distal end (farthest from insertion point) and each end is coupled to a hub. In some embodiments, the extending element is releasably coupled to a hub on at least one end. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or a length of hypotube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypotube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK), polyether ketone ketone (PEKK), polyethylenimine (PEI), or polyimide (PI). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may include a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, insert catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 can be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 can be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The insert catheter hub 28 and insert catheter 29 (which can be, for example, any of the insert catheters described with reference to FIGS. 9A-14E), and guidewire hub 26 and guidewire 27 are illustrated as residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices are configured in an access assembly for aortic access, following coupling of the access assembly to the corresponding carriages beneath the sterile barrier. The access assembly may be preassembled with the guidewire fully advanced through the insert catheter, which is in turn fully advanced through the guide catheter. In embodiments in which the insert catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 can be the distal most hub. Insert catheter hub 28 can be positioned proximally of the guide catheter hub, so that the insert catheter 29 can extend distally through the guide catheter. The guidewire hub 26 can be positioned most proximally, in order to allow the guidewire 27 to advance through the insert catheter 29 and guide catheter 31.

A procedure assembly is illustrated in FIG. 3F following introduction of the procedure assembly through the guide catheter 31 that was used to achieve supra-aortic access. In this implementation, guide catheter 31 can remain the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

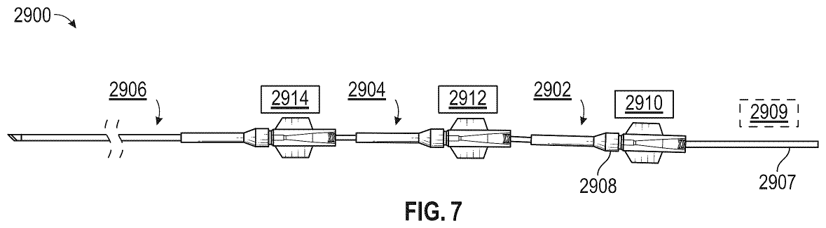

As is discussed in greater detail in connection with FIG. 7, the multi catheter stack may be utilized to achieve both access and the intravascular procedure without the need for catheter exchange. This may be accomplished in either a manual or a robotically driven procedure. In some embodiments, a suitable catheter stack each having certain dimensions, along with a fluidics system configured to perform contrast injections can utilize the configuration of the catheter stack disclosed herein can facilitate access, intravascular procedure, and contrast injection without the need for catheter exchange. In one example, the guide catheter 31 may include a catheter having an inner diameter of at least about 0.08 inches and in one implementation about 0.088 inches. The first procedure catheter 120 may include a catheter having an inner diameter within the range of from about 0.065 inches to about 0.075 inches and in one implementation catheter 120 has an inner diameter of about 0.071 inches. The second procedure catheter 124 may be an insert catheter having an OD sized to permit advance through the first procedure catheter 120. The second procedure catheter may be steerable, having a deflection control 2908 configured to laterally deflect a distal end of the catheter. The second procedure (or insert) catheter 120 may also have an inner lumen sized to allow an appropriately sized guidewire to remain inside the second procedure catheter while performing contrast injections through the second procedure catheter. Additional details regarding a method for controlling hub assemblies can be found in U.S. patent application Ser. No. 19/228,468, entitled METHOD FOR ROBOTICALLY CONTROLLING INTERVENTIONAL DEVICE ASSEMBLY, filed Jun. 4, 2025, and in U.S. patent application Ser. No. 18/525,267, entitled METHOD FOR ROBOTICALLY CONTROLLING SUBSETS OF INTERVENTIONAL DEVICE ASSEMBLY, filed Nov. 30, 2023, the entirety of which are hereby incorporated by reference herein.

In certain embodiments, the catheter 31 may be a “large bore” catheter (e.g., a guide catheter) having an inner diameter of at least about 0.075 inches or at least an inner diameter of about 0.080 inches. The catheter 120 may be an aspiration catheter having an inner diameter within the range of from about 0.060 to about 0.075 inches. The catheter 124 may be a steerable catheter (e.g., an insert or access catheter) with a deflectable distal tip, having an inner diameter within the range of from about 0.025 to about 0.050 inches. The guidewire (or guiding element) 27 may have an outer diameter within the range of from about 0.014 to about 0.020 inches. In one example, the catheter 31 may have an inner diameter of about 0.088 inches, the catheter 120 an inner diameter of about 0.071 inches, the catheter 124 an inner diameter of about 0.035 inches, and the guidewire 27 may have an outer diameter of about 0.018 inches. In another example, the catheter 31 may have an inner diameter of about 0.088 inches, the catheter 120 an inner diameter of about 0.071 inches, the catheter 124 an inner diameter of about 0.045 inches, and the guidewire 27 may have an outer diameter of about 0.018 inches.

In one implementation, a preassembled access assembly (guide catheter, insert catheter, and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure. Additional details regarding a method for controlling hub assemblies can be found in U.S. patent application Ser. No. 18/060,935, entitled METHOD OF PRIMING AN INTERVENTIONAL DEVICE ASSEMBLY, filed Dec. 1, 2022, the entirety of which is hereby incorporated by reference herein.

FIGS. 3G-3K illustrate embodiments of an alternate sterile barrier having a convex drive surface (e.g., a convex, crowned road like drive surface). FIG. 3G is a cross-sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

FIGS. 3I, 3J, and 3K illustrate a sectional perspective view, a cross-sectional view, and a top sectional view, respectively, of a proximal end of the sterile barrier 232. As shown, in FIGS. 3I-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 3I-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices. In some embodiments, any of the channels or wells described herein may not be part of the sterile barrier, but may instead be part of the drive table positioned below the sterile barrier.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width x₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h₁ of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively, or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician. A vertical configuration may also provide for a lower axis of catheter travel closer to a patient without adding standoff height to the drive system 18.

In some embodiments, the drive system 18 may be positioned, during part of, or the entirety of, an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively, or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

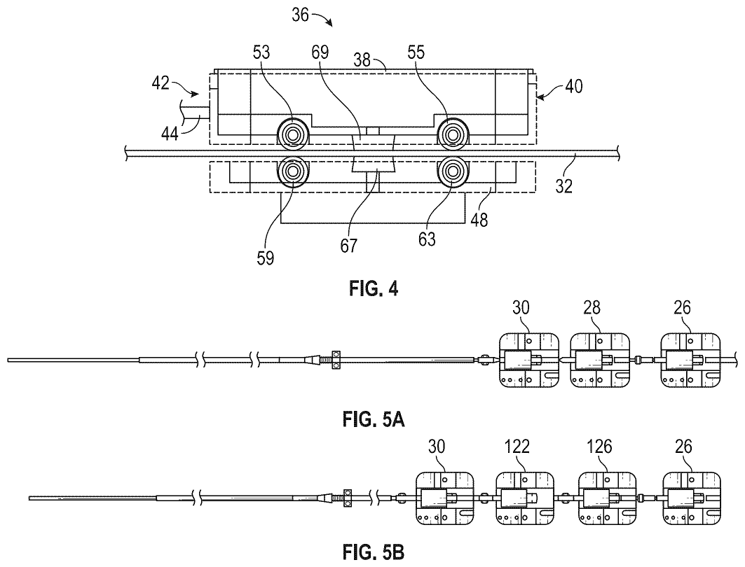

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or controller manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4. In other embodiments, any of the interventional devices and/or hubs described herein may be coupled to a carriage or hub adapter via a mechanical coupling.

Additional details regarding a magnetic coupling through a sterile barrier can be found in U.S. patent application Ser. No. 18/678,766, entitled MAGNETIC COUPLING THROUGH A STERILE FIELD BARRIER, filed May 30, 2024, the entirety of which is hereby incorporated by reference herein.

Certain embodiments of hub assemblies described herein, such as hub assembly (“hub”) 36 shown in FIG. 4, include a housing (e.g., housing 38) for coupling an interventional device thereto, components (e.g., rollers 53 and 55) for directly coupling to and moving along a drive table, and magnet(s) (e.g., magnet 69) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure).

An arrangement of a hub assembly having a hub that is releasably couplable to mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The robotic drive systems described herein can relate to various embodiments of systems that include a hub, or a hub and a mount, regardless of whether they are described in reference to a hub, or a hub and mount, unless explicitly indicated or indicated by context. In some embodiments, a mount may be magnetically coupled to hub adapter across a sterile barrier prior to coupling a hub to the mount, for example, when preparing the drive table for a medical procedure.

As described herein, the hub assemblies can include intravascular devices that can access the vascular system of a patient via at least one artery and/or vein (e.g., the femoral artery) and be driven within the vascular system to perform a vascular procedure.

### Example Catheters/Catheter Stacks

Any of the catheters illustrated, for example, in FIGS. 5A, 5B, 6, and 9A-12D generally include an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

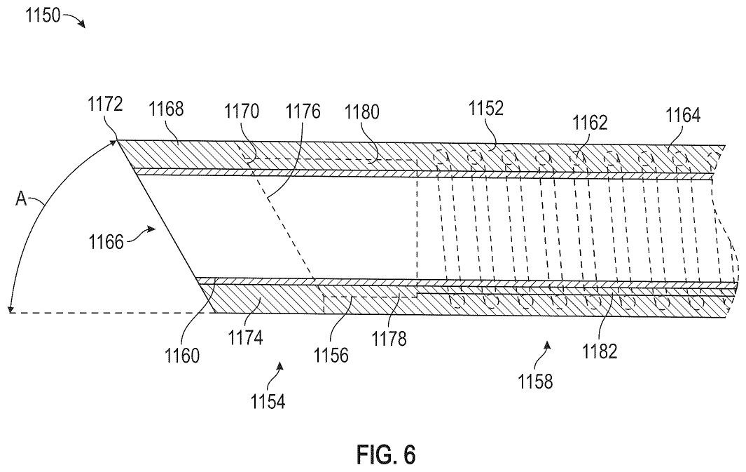

Any of the catheters disclosed herein may be provided with an inclined distal tip. Referring to FIG. 6, distal catheter tip 1150 can include a tubular body 1152 which includes an advance segment 1154, a marker band 1156, and a proximal segment 1158. An inner tubular liner 1160 may extend throughout the length of the distal catheter tip 1150, and may include dip coated or extruded PTFE or other lubricious material.

A reinforcing element 1162 such as a braid and/or spring coil is embedded in an outer jacket 1164, which may extend the entire length of the catheter. Additional details of an example catheter with a braid will be described below with reference to FIG. 9A-12D. In some embodiments, such as shown in FIGS. 9A-12D, a portion of the braid can be annealed.

The advance segment 1154 can terminate distally in an angled face 1166, to provide a leading side wall portion 1168 having a length measured between the distal end 130 of the marker band 1156 and a distal tip 1172. In some embodiments, the entire distal tip may be shaped to avoid snagging the tip in areas of arterial bifurcation. A trailing side wall portion 1174 of the advance segment 1154, has an axial length in the illustrated embodiment of approximately equal to the axial length of the leading side wall portion 1168 as measured at approximately 180 degrees around the catheter from the leading side wall portion 1168. The leading side wall portion 1168 may have an axial length within the range of from about 0.1 millimeters to about 5 millimeters and generally within the range of from about 1 to 3 millimeters. The trailing side wall portion 1174 may be equal to or at least about 0.1 or 0.5 or 1 millimeter or 2 millimeters or more shorter than the axial length of the leading side wall portion 1168, depending upon the desired performance.

The angled face 1166 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105 percent, and no more than about 130 percent, in some implementations within the range of from about 110 percent and about 125 percent, and in one example is about 115 percent of the area of the corresponding circular port (angle A is 90 degrees).

In the illustrated embodiment, the axial length of the advance segment is substantially constant around the circumference of the catheter, so that the angled face 1166 is approximately parallel to the distal surface 1176 of the marker band 1156. The marker band 1156 has a proximal surface approximately transverse to the longitudinal axis of the catheter, producing a marker band 1156 having a right trapezoid configuration inside elevational view. A short sidewall 1178 is rotationally aligned with the trailing side wall portion 1174 and has an axial length within the range of from about 0.2 millimeters to about 4 millimeters, and typically from about 0.5 millimeters to about 2 millimeters. An opposing long sidewall 1180 is rotationally aligned with the leading side wall portion 1168. Long sidewall 1180 of the marker band 1156 is generally at least about 10 percent or 20 percent longer than short sidewall 1178 and may be at least about 50 percent or 70 percent or 90 percent or more longer than short sidewall 1178, depending upon desired performance. Generally, the long sidewall 1180 can have a length of at least about 0.5 millimeters or 1 millimeter and less than about 5 millimeters or 4 millimeters.

In some embodiments, such as shown in FIGS. 9A-12D, a distal catheter tip may not include an advanced segment that has an angled face.

The marker band may be a continuous annular structure, or may have at least one and optionally two or three or more axially extending slits throughout its length. The slit may be located on the short sidewall 1178 or the long sidewall 1180 or in between, depending upon desired bending characteristics. The marker band may include any of a variety of radiopaque materials, such as a platinum/iridium alloy, with a wall thickness preferably no more than about 0.003 inches and in one implementation is about 0.001 inches.

The fluoroscopic appearance of the marker bands may be unique or distinct for each catheter size or type when a plurality of catheters is utilized so that the marker bands can be distinguishable from one another by a software algorithm. Distinguishing the marker bands of a plurality of catheters may be advantageous when the multiple catheters are used together, for example, in a multi catheter assembly or stack as described herein. In some embodiments, the marker band of a catheter may be configured so that a software algorithm can detect motion of the catheter tip.

The marker band zone of the assembled catheter may have a relatively high bending stiffness and high crush strength, such as at least about 50 percent or at least about 100 percent less than proximal segment 18 but generally no more than about 200 percent less than proximal segment 1158. The high crush strength may provide radial support to the adjacent advance segment 1154 and particularly to the leading side wall portion 1168, to facilitate the functioning of distal tip 1172 as an atraumatic bumper during transluminal advance and to resist collapse under vacuum. The proximal segment 1158 preferably has a lower bending stiffness than the marker band zone, and the advance segment 1154 preferably has even a lower bending stiffness and crush strength than the proximal segment 1158.

The advance segment 1154 may include a distal extension of the outer tubular jacket 1164 and optionally the inner liner 1160, without other internal supporting structures distally of the marker band 1156. Outer jacket 1164 may include extruded polyurethane, such as Tecothane® or NEUsoft™. The advance segment 1154 may have a bending stiffness and radial crush stiffness that is no more than about 50 percent, and in some implementations no more than about 25 percent or 15 percent or 5 percent or less than the corresponding value for the proximal segment 1158.

The catheter may further include an axial tension element or support such as a ribbon or one or more filaments or fibers for increasing the tension resistance and/or influencing the bending characteristics in the distal zone. The tension support may include one or more axially extending mono strand or multi strand filaments. The one or more tension element 1182 may be axially placed inside the catheter wall near the distal end of the catheter. The one or more tension element 1182 may serve as a tension support and resist tip detachment or elongation of the catheter wall under tension (e.g., when the catheter is being proximally retracted through a kinked outer catheter or tortuous or narrowed vasculature).

At least one of the one or more tension element 1182 may proximally extend along the length of the catheter wall from within about 1.0 centimeters from the distal end of the catheter to less than about 10 centimeters from the distal end of the catheter, less than about 20 centimeters from the distal end of the catheter, less than about 30 centimeters from the distal end of the catheter, less than about 40 centimeters from the distal end of the catheter, or less than about 50 centimeters from the distal end of the catheter.

The one or more tension element 1182 may have a length greater than or equal to about 40 centimeters, greater than or equal to about 30 centimeters, greater than or equal to about 20 centimeters, greater than or equal to about 10 centimeters, or greater than or equal to about 5 centimeters.

At least one of the one or more tension element 1182 may extend at least about the most distal 50 centimeters of the length of the catheter, at least about the most distal 40 centimeters of the length of the catheter, at least about the most distal 30 centimeters or 20 centimeters or 10 centimeters of the length of the catheter.

In some implementations, the tension element extends proximally from the distal end of the catheter along the length of the coil 24 and ends proximally within about 5 centimeters or 2 centimeters or less either side of a transition between a distal coil and a proximal braid. The tension element may end at the transition without overlapping with the braid.

The one or more tension element 1182 may be placed near or radially outside the inner liner 1160. The one or more tension element 1182 may be placed near or radially inside the braid and/or the coil. The one or more tension element 1182 may be carried between the inner liner 1160 and the helical coil and may be secured to the inner liner or other underlying surface by an adhesive prior to addition of the next outer adjacent layer such as the coil. Preferably, the tension element 1182 is secured to the marker band 1156 such as by adhesives or by mechanical interference. In one implementation, the tension element 1182 extends distally beyond the marker band on a first (e.g., inside) surface of the marker band, then wraps around the distal end of the marker band and extends along a second (e.g., outside) surface in either, or both, a proximal inclined or circumferential direction to wrap completely around the marker band.

When more than one tension element 1182 or filament bundles are spaced circumferentially apart in the catheter wall, the tension elements 1182 may be placed in a radially symmetrical manner. For example, the angle between two tension elements 1182 with respect to the radial center of the catheter may be about 180 degrees. Alternatively, depending on desired clinical performances (e.g., flexibility, trackability), the tension elements 1182 may be placed in a radially asymmetrical manner. The angle between any two tension elements 1182 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 1182 may include materials such as Vectran®, Kevlar®, Polyester®, Spectra®, Dyneema®, Meta-Para-Aramide®, or any combinations thereof. At least one of the one or more tension element 1182 may include a single fiber or a multi-fiber bundle, and the fiber or bundle may have a round or rectangular (e.g., ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 1182, as measured in the radial direction, may be no more than about 2 percent, 5 percent, 8 percent, 15 percent, or 20 percent of that of the catheter 10.

The cross-sectional dimension of the one or more tension element 1182, as measured in the radial direction, may be no more than about 0.03 millimeters (about 0.001 inches), no more than about 0.0508 millimeters (about 0.002 inches), no more than about 0.1 millimeters (about 0.004 inches), no more than about 0.15 millimeters (about 0.006 inches), no more than about 0.2 millimeters (about 0.008 inches), or about 0.38 millimeters (about 0.015 inches).

The one or more tension element 1182 may increase the tensile strength of the distal zone of the catheter before failure under tension (e.g., marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

In some implementations, such as shown in FIGS. 9A-11D, the distal catheter tip may not include a tension element.

FIG. 7 illustrates a side elevational schematic view of a multi catheter interventional device assembly 2900 for combined supra-aortic access and/or neurovascular site access and procedure (e.g., aspiration), as described herein. The multi catheter assembly 2900 may be configured for either a manual or a robotic procedure.

The interventional device assembly 2900 includes an insert or access catheter 2902 (such as the example insert catheters described with reference to FIGS. 9A-14E), a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two-stage procedure disclosed previously, a first stage for achieving supra-aortic access, includes mounting an insert catheter, guide catheter and guidewire to the support table. Upon gaining supra-aortic access, the insert catheter and guidewire were typically removed from the guide catheter. Then, a second catheter assembly is introduced through the guide catheter after attaching a new guidewire hub and a procedure catheter hub to the corresponding drive carriage on the support table.

The single multi catheter assembly 2900 of FIG. 7 is configured to be operated without having to remove hubs and catheters, and without the addition of additional assemblies and/or hubs. Thus, the multicomponent access and procedure configuration of assembly 2900 may utilize a guidewire 2907 manufactured (configured) to function as both an access guidewire and a navigation guidewire to allow for sufficient access and support, and navigation to the particular distal treatment site. In a non-limiting example configured for robotic implementation, a catheter assembly 2900 may include a guidewire hub (e.g., guidewire hub 2909 or guidewire hub 26 (e.g., FIG. 3A) positioned on a drive table and to the right of catheter 2902 relative to the orientation of FIG. 7), an insert or access catheter hub 2910, a procedure catheter hub 2912, a guide catheter hub 2914 and corresponding catheters. In certain embodiments, one or more of the hubs may include, or be coupled to, a hemostasis valve (e.g., a rotating hemostasis valve) to accommodate introduction of interventional devices therethrough, and/or introduction of fluids (e.g., saline, contrast). In certain embodiments, one or more of the hubs may include, or be coupled to, a fluidic system (which may include a hemostasis valve) for the introduction of a fluid (e.g., saline, contrast) and the application of a vacuum for aspiration functions. Additional examples regarding hemostasis valves, fluidic systems, and aspiration systems are included in U.S. patent application Ser. No. 17/879,614, entitled Multi Catheter System With Integrated Fluidics Management, filed Aug. 2, 2022, and hereby expressly incorporated by reference in its entirety herein.

Once access above the aortic arch has been achieved, the insert or access catheter 2902 (associated with insert catheter hub 2910) may be “parked,” for example, in the vicinity of a carotid artery ostia, and the remainder or a subset of the catheter assembly may be guided more distally toward a particular site (e.g., a clot site, a surgical site, a procedure site, etc.).

In some embodiments, other smaller procedure catheters may also be added and used at the site. In some implementations, for catheter assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of, or in addition to, the catheter 2904. The insert catheter 2902 may have a distal deflection zone and can function to access a desired ostium. In some implementations, insert catheters such as shown in FIG. 9A-11D may include a pre-shaped distal tip to facilitate access of a desired ostium. Either manual manipulation or robotic manipulation of the multi-catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations. Since each catheter can have its own stiffness profile relative to length, the position and superposition of the catheters can be adjusted to find the corresponding optimal stiffness profile to help navigate the catheter stack past various anatomical obstacles.

In a non-limiting example, the catheter assembly 2900 may be used for a diagnostic angiogram procedure. In some embodiments, the assembly 2900 may include only the guidewire 2907 and insert catheter 2902 (in the form of a diagnostic angiographic catheter) for performing the diagnostic angiogram procedure or only the guidewire 2907 and the insert catheter 2902 may be utilized during the procedure. Alternatively, the guide catheter 2906 and procedure catheter 2904 may be retracted proximally to expose the distal end of the insert catheter 2902 (e.g., a few centimeters of the distal end of the insert catheter) to perform the diagnostic angiography.

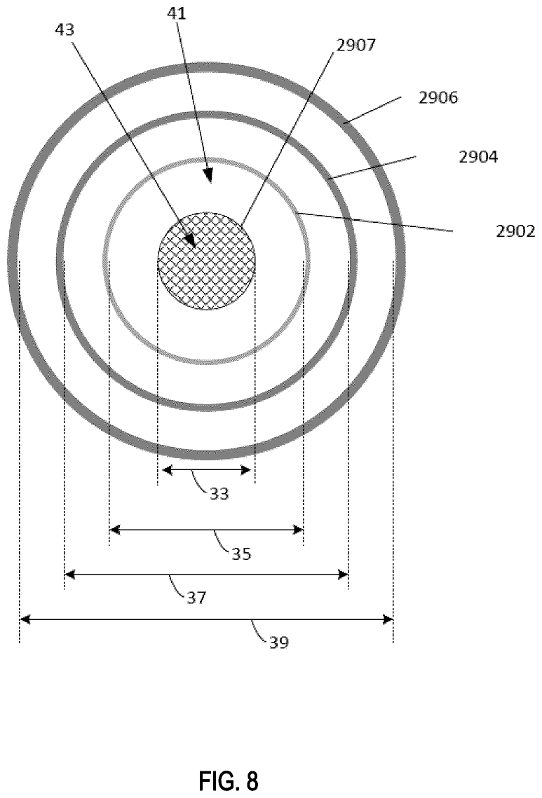

As shown in FIG. 7, the guide catheter 2906, procedure catheter 2904, insert catheter 2902, and guidewire (or guiding element) 2907 can be arranged concentrically. In certain embodiments, a multi catheter interventional device assembly having a plurality of concentrically arranged catheters may be referred to as a multi catheter stack. FIG. 8 illustrates a representation of a distal end view of the guide catheter 2906, the procedure catheter 2904, the insert catheter 2902 (such as the example insert catheters described with reference to FIGS. 9A-14E), and the guidewire 2907 illustrating a concentric arrangement. For example, as illustrated in FIG. 8, the outside diameter 33 of the guidewire 2907 is smaller than the inside diameter 35 of the insert catheter 2902, and the system is configured such that the guidewire 2907 can be at least partially positioned in the lumen of the insert catheter 2902. The outside diameter of the insert catheter 2902 is smaller than the inside diameter 37 of the procedure catheter 2904, and the system is configured such that the insert catheter 2902 can be at least partially positioned in the lumen of the procedure catheter 2904. The outside diameter of the procedure catheter 2904 is smaller than the inside diameter 39 of the guide catheter 2906, and the system is configured such that the procedure catheter 2904 can be at least partially positioned in the lumen of the guide catheter 2906. In certain embodiments, the guide catheter 2906 may be a ‘large bore’ guide catheter having a bore (inner) diameter 39 of at least about 0.075, or at least about 0.080 inches in diameter. In an example, the inner diameter 39 of the guide catheter 2906 is 0.088″. The procedure catheter 2904 may be an aspiration catheter having an inner diameter 37 within the range of from about 0.060 to about 0.075 inches. The insert catheter 2902 may be a steerable catheter with a deflectable distal tip, having an inner diameter 35 within the range of from about 0.025 to about 0.050 inches. In some examples, the insert catheter 2902 has an inner diameter 35 of between about 0.045″ and 0.049″. This configuration allows for an effective cross-sectional area of the lumen of the insert catheter for fluid communication when a suitably sized guidewire 2907 is positioned in the insert catheter, as described further herein. In some examples, a suitably-sized guidewire 2907 has an outer diameter 35 within the range of from about 0.014 to about 0.020 inches (0.020″). In some examples, the guidewire 2907 may have an outer diameter 35 of 0.014″, 0.015″, 0.016″, 0.017″, 0.018″, 0.019″, 0.020″, 0.021″, 0.022″, 0.023″, or 0.024″ plus or minus 0.0005″. However, a guidewire of a smaller diameter (e.g., 0.014″-0.020″) is preferred to provide the desired effective cross-sectional area of an insert catheter for fluid injection when the guidewire is positioned at least partially in the insert catheter lumen. In one example of a system configuration where the system can inject contrast media (or another fluid) into a patient through an insert catheter 2902 while a guidewire 2907 is positioned partially or fully in the lumen of the insert catheter 2902, the guide catheter 2906 may have an inner diameter 39 of about 0.088 inches, the procedure catheter 2904 an inner diameter 37 of about 0.071 inches, the insert catheter 2902 an inner diameter 35 of about 0.035-0.048 inches, and the guidewire 2907 may have an outer diameter 33 of about 0.018 inches. In an example, the guide catheter 2906 can have an outside diameter of about 0.110 inches, the procedure catheter 2904 can have an outside diameter of about 0.083 inches, and the insert catheter 2902 can have an outside diameter of about 0.061 inches to about 0.070 inches.

In some embodiments, the insert catheter 2902 may have an inner diameter 35 of about between 0.046 to 0.047 inches, and the guidewire 2907 may have an outer diameter 33 of less than or equal to 0.024 inches, for example, an outer diameter 33 in the range between about 0.014 and about 0.024 inches. These configuration provides sufficient annular space within the lumen of insert catheter 2902 for contrast media to propagate through the lumen of the insert catheter 2902 so the guidewire 2907 may remain in place while contrast media is injected into the catheter assembly 2900. This advantageously shortens the time of the overall procedure by eliminating the need to remove the guidewire 2907 each time contrast media is to be injected to create enough annular space within the lumen of the insert catheter 2902 for the contrast media to flow through the lumen of the insert catheter 2902. For example, these configurations provide allow for contrast to flow inside a catheter at a flow rate of about 3 cc's per second, at a pressure not to exceed 400 psi. In some examples, such a catheter is between about 100 cm and 160 cm in length. Additionally, this configuration lowers the risk of air embolisms associated with the repeated removal and re-insertion of the guidewire 2907 from, and into, the catheter assembly 2900.

As described further in reference to the examples/trails, to provide a desired fluid flow rate through the insert catheter 2902 while the guidewire 2907 is positioned within the insert catheter 2902, a certain effective cross-sectional area 41 of the insert catheter 2902 should be available to communicate fluid from the proximal end of the catheter to the distal end and out of the insert catheter. In reference to FIG. 8, the effective cross-sectional area 41 of the insert catheter 2902 (i.e., the space in the lumen of the insert catheter 2902 where fluid can be communicated when the guidewire 2907 is positioned in the lumen) is determined by subtracting the cross-sectional area 43 of the guidewire 2907 from the cross-sectional area of the insert catheter 2902. Through testing related to designed configurations of providing fluid communication through the access catheter with the guide element positioned in its lumen, working pressures can be determined that are feasible to generate with a contrast pump and the fluidics system. Typically, the working pressure is a relatively high pressure. In some examples, the high pressure can be less than or equal to about 400 PSI. For example, above about 200 PSI, above about 250 PSI, above about 300 PSI, between about 350 PSI and about 400 PSI, between about 300 PSI and about 400 PSI, between about 250 PSI and about 400 PSI, between about 200 PSI and about 400 PSI, or between about 150 PSI and about 400 PSI. In some embodiments, where the portion of a fluidics system has been designed to use higher pressures, the working pressure can be greater than about 400 PSI, or even greater than about 500 PSI. Typically, the pressure is on the higher side of the range of about 250-about 400 PSI for proper operation (for example, a quick injection of contrast to facilitate a medical practitioner to see the patient's anatomy).

In some embodiments, it was determined that at these working pressures, an effective cross-sectional area 41 in a lumen of at least about 0.001 square inches can be advantageous. In an example, it was determined that at these pressure ranges, an effective cross-sectional area 41 of at least about 0.001257 square inches (e.g., about 0.001 square inches) can be advantageous. In another example, an effective cross-sectional area 41 of at least 0.001407 square inches can be advantageous to produce a desired contrast fluid flow at less than or equal to about 400 psi. For the example where it was determined that an effective cross-sectional area 43 of at least about 0.001257 square inches was desired, various dimensions of the outside diameter 33 of the guidewire 2907 and the inside diameter 35 of the insert catheter 2902 can be utilized to achieve an effective cross-sectional area of at least about 0.001257 square inches as shown in Table 1A below, which shows examples of determined effective cross-sectional areas of the insert catheter for various insert catheter inner diameter dimensions and various guidewire or guiding element (GE) outer diameter dimensions where the shaded cells indicate effective cross-sectional areas that fall below the threshold of 0.001257 square inches. In an example, as illustrated in Table 1A, an insert catheter ID of 0.045″ and a guidewire OD of 0.020″ provides an effective cross-sectional area of the insert catheter of 0.001276 square inches, which is acceptable (un-shaded) because it is above the threshold value of 0.001257 square inches. In another example, as illustrated in Table 1A, an insert catheter ID of 0.045″ and a guidewire OD of 0.021″ provides an effective cross-sectional area of the insert catheter of 0.001244 square inches which is unacceptable (shaded) because it is below a (predetermined) threshold value of 0.001257 square inches. For configurations where it is determined that an effective cross-sectional area of at least about 0.001 square inches is needed to produce a desired flow rate (e.g., of about 1 mL/sec., 2 mL/sec., or 3 mL/sec.), all of the configurations shown in Table 1A with access catheter inner diameters (ID) of 0.045″ to 0.055″ and guiding element outer diameters (OD) of 0.014″ to 0.025″ result in an effective cross-sectional area of the access catheter of at least 0.001 square inches. Although Table 1A below illustrates some examples, others example configurations are also possible that meet this threshold. A controller can cause the fluidics system to inject contrast through the procedure catheter 2904 when the insert catheter 2902 and/or the guidewire 2907 is positioned in the lumen of the procedure catheter 2904. A controller can cause the fluidics system to inject contrast though the guide catheter 2906 when the procedure catheter 2904 is positioned in the lumen of the guide catheter 2906 and/or the insert catheter 2902 and/or the guidewire is positioned in the lumen of the guide catheter 2906 because cross-sectional areas 45 and 47 are also greater than at least about 0.001257.

TABLE 1A
EFFECTIVE CROSS-SECTIONAL
AREA OF INSERT CATHETER

| | Insert catheter Inner Diameter (inches) | | | | |
|---|---|---|---|---|---|
| GE OD | 0.045″ | 0.046″ | 0.047″ | 0.048″ | 0.049″ |
| 0.014″ | 0.001436 | 0.001508 | 0.001581 | 0.001656 | 0.001732 |
| 0.015″ | 0.001414 | 0.001485 | 0.001558 | 0.001633 | 0.001709 |
| 0.016″ | 0.001389 | 0.001461 | 0.001534 | 0.001608 | 0.001685 |
| 0.017″ | 0.001363 | 0.001435 | 0.001508 | 0.001583 | 0.001659 |
| 0.018″ | 0.001336 | 0.001407 | 0.001480 | 0.001555 | 0.001631 |
| 0.019″ | 0.001307 | 0.001378 | 0.001451 | 0.001526 | 0.001602 |
| 0.020″ | 0.001276 | 0.001348 | 0.001421 | 0.001495 | 0.001572 |
| 0.021″ | 0.001244 | 0.001316 | 0.001389 | 0.001463 | 0.001539 |
| 0.022″ | 0.001210 | 0.001282 | 0.001355 | 0.001429 | 0.001506 |
| 0.023″ | 0.001175 | 0.001246 | 0.001319 | 0.001394 | 0.001470 |
| 0.024″ | 0.001138 | 0.001210 | 0.001283 | 0.001357 | 0.001433 |
| 0.025″ | 0.001100 | 0.001171 | 0.001244 | 0.001319 | 0.001395 |

In some embodiments, the length of the insert catheter 2902 may be between about 100 and 193 centimeters. In certain embodiments, a wall of the insert catheter 2902 surrounding the lumen may include a braided reinforcement layer and an interior liner (e.g., PEBAX). The braided reinforcement layer may include stainless metal ribbon wire in a tight braid pattern. In an example, the metal ribbon wire can have a cross-sectional dimension of about 0.002″×about 0.005″. In other embodiments, the braided reinforcement layer may include a stainless metal round wire having a 0.002 inch diameter in a tight braid pattern (e.g., a 1:1 braid pattern, or a 2:2 braid pattern). In various examples, the ribbon material could be made from, or include, stainless steel or other materials, including titanium, CoCr alloys, Elgiloy, Hastelloy (Hastelloy alloy), or the like.

Table 1B (below) illustrates an example of effective cross-sectional areas of a procedure catheter (“P Cath”) and a guide catheter (“G Cath”) that are in a concentric stack when an elongated medical device (“EMD”) (e.g., a guiding element or a catheter) is in the lumen of the procedure catheter or the guide catheter, for certain embodiments. The EMD outer diameters (“OD”) are shown in two columns to the left of the P Cath and G Cath columns, the EMD outer diameters in the range of 0.014 inches to and the 0.083 inches. As illustrated in Table 1B, for a procedure catheter having an ID of 0.071 inches, an EMD having an OD of 0.014″ to 0.061 inches results in an effective diameter (in the lumen of the procedure catheter) of at least about 0.001 square inches. These are examples of configurations of a concentric stack of EMDs that can be used to provide a desired flow rate of at least about 1 mL/second. Table 1B shows that an EMD having an OD of 0.068 inches falls below the 0.001 square inches “threshold”—in examples with this configuration a higher contrast pump working pressure may be needed to produce the desired contrast flow rate (which as indicated above, such a higher working pressure may not be possible or desirable due to the design of the contrast pump and the rest of a fluidics system). Also as illustrated in Table 1B, for a guide catheter having an ID of 0.088 inches, an EMD having an OD of 0.014 inches to 0.08 inches results in an effective diameter (in the lumen of the guide catheter) of at least about 0.001 square inches. Table 1B shows that an EMD having an OD of 0.083 inches falls below the 0.001 square inches “threshold”—in examples with this configuration a higher contrast pump working pressure may be needed to produce the desired contrast flow rate (which as indicated above, such a higher working pressure may not be possible or desirable due to the design of the contrast pump and the rest of a fluidics system).

TABLE 1B
EFFECTIVE CROSS-SECTIONAL AREA OF PROCEDURE
CATHETER AND A GUIDE CATHETER

Catheter Inner Diameter (inches)

| | P Cath | G Cath |
|---|---|---|
| EMD OD | 0.071″ | 0.088″ |
| 0.014″ | 0.003805 | 0.005928 |
| 0.015″ | 0.003782 | 0.005905 |
| 0.016″ | 0.003758 | 0.005881 |
| 0.017″ | 0.003732 | 0.005855 |
| 0.018″ | 0.003705 | 0.005828 |
| 0.019″ | 0.003676 | 0.005799 |
| 0.020″ | 0.003645 | 0.005768 |
| 0.021″ | 0.003613 | 0.005736 |
| 0.022″ | 0.003579 | 0.005702 |
| 0.023″ | 0.003544 | 0.005667 |
| 0.024″ | 0.003507 | 0.005630 |
| 0.025″ | 0.003468 | 0.005591 |
| 0.061″ | 0.001037 | 0.003160 |
| 0.068″ | 0.000328 | 0.002450 |
| 0.08″ | — | 0.001056 |
| 0.083″ | — | 0.000672 |

Example Insert Catheter with Pre-Shaped Distal Tip

Additional details of an insert catheter with a pre-shaped distal tip will be described with reference to FIGS. 9A-14E. The insert catheters as shown in FIGS. 9A-14E can incorporate any features of the other catheters described elsewhere in the present disclosure, including but not limited to the catheter in FIG. 6 and/or other insert catheters disclosed herein. The insert catheter can have a size of 5 Fr (that is, having an average outer diameter of 0.066″). In some examples, the maximum outer diameter of the insert catheter is 0.068″. The insert catheter can be used in percutaneous interventional procedures to atraumatically select appropriate ostium access and guide aspiration devices for stroke treatment. FIGS. 9A-9E illustrate various features of an example insert catheter 1400. In some implementations, the insert catheter 1400 can include a single lumen 1412. The single lumen can accommodate a guidewire, as described elsewhere in the present disclosure.

To implement the features of the catheter stacks as disclosed herein, for example, the ability to inject a contrast agent via the lumen of the insert catheter without having to remove the guidewire, the insert catheter, such as the example catheters shown in FIGS. 9A-12D may include an inner diameter or a bore size within the range as shown in the table above (for example, from 0.045″ to 0.049″, or about 0.045″ to about 0.047″, or 0.046″). Therefore, for a 5 Fr catheter, a wall thickness of the insert catheter disclosed herein can have a wall thickness of about 0.0085″ to about 0.0115″, or about 0.0095″ to about 0.0110″, or about 0.0100″.

The thin wall of the insert catheter can buckle or break more easily, or have a reduced column strength, and/or have more pronounced discontinuity in stiffness along a length of the catheter than a catheter with a thicker wall. To reinforce the catheter and/or improve its column strength, the insert catheter can include braids substantially throughout the entire length (for example, from the proximal end up to about 3.5 cm, about 3.2 cm, about 3.0 cm, about 2.0 cm, about 1.5 cm, about 1.0 cm, about 0.5 cm, or about 0.25 cm from the distal end of the catheter or between about 0.25 cm and 3.5 cm or between about 0.5 cm and 2.0 cm). As shown in FIGS. 9A-9E, the insert catheter 1400 can include a braid 1404 that extends continuously from the proximal end (see FIG. 9E) up to a distal most portion of the catheter 1400 (see FIG. 9C).

The braid 1404 can be made of metal wires. The metal wires can be stainless steel wires or other suitable metal wires. The wires can have a diameter between about 0.001″ to about 0.003″, or about 0.002″. The braid 1404 can have a diamond (for example, 2 over 2 under) pattern, or other suitable patterns. In some implementations, the braid 1404 can have a braid density of about 30 picks per inch (PPI) to about 110 PPI, or about 40 PPI to about 100 PPI, or about 60 PPI to about 80 PPI, or about 40 PPI, or about 70 PPI. The braid 1404 can include 16 carriers or 32 carriers. In some implementations, the braid 1404 can include 32 carriers. In some implementations, the braid 1404 can be a straight braid (i.e., no braid transfer). The 32-carrier, straight braid design can improve shape stability during ostial engagement. The 32-carrier, straight braid design can additionally allow the catheter 1400 to more easily straighten upon guidewire advancement through the lumen 1412 of the catheter 1400. The 32-carrier, straight braid design can further provide a more robust transition between a distal polymer portion and a proximal hypotube portion, which will be described in greater detail below.

The distal most portion of the catheter 1400 can include a soft distal tip 1402 terminating at a distal end of the catheter 1400 and a radiopaque marker 1406 immediately proximal to the soft distal tip 1402. In other words, the soft distal tip 1402 and the radiopaque marker 1406 do not include a braid. The soft distal tip 1402 can have a length of about 1.97 mm to about 2.03 mm, or about 2.00 mm. The soft distal tip 1402 can facilitate atraumatic advancement of the insert catheter. The radiopaque marker 1406 can have a length of about 0.99 mm to about 1.04 mm, or about 1.01 mm. In some implementations, the soft distal tip 1402 can have a durometer value of about 40D. The soft distal tip 1402 can include a first polymer, such as Polyethylene terephthalate (PET) or another suitable polymer of a similar hardness as the PET. The first polymer can not only form at least partially the soft distal tip 1402, but also extend over the radiopaque marker 1406 and a distal segment of the braid 1404.

As shown in FIGS. 9A-9C, the first polymer (e.g., PET) in the soft tip 1402 can be covered by a layer of a second polymer of a first stiffness 1408 on a radially outer surface of the first polymer segment and an inner liner layer 1414 defining the lumen 1412 of the catheter 1400 on a radially inner surface of the first polymer segment. In some implementations, the second polymer can include a thermoplastic elastomer, such as Polyether block amide (also referred to by the trade name Pebax™). As shown in FIG. 9C, the second polymer of the first stiffness 1408 can form the soft distal tip 1402 and extend over the radiopaque marker 1406 and a distal segment of the braid 1404 that is contained by the first polymer. In other words, the first polymer segment and the second polymer of the first stiffness 1408 can have the same length. In some implementations, the second polymer of the first stiffness 1408 and/or the first polymer segment can have a length of about 4.83 mm to about 5.33 mm, or about 5 mm. As shown in FIGS. 9D-9E, the inner liner layer 1414 can extend throughout the length of the catheter 1400. The liner layer 1414 can be made of a polymer, which may optionally be the same as the second polymer, such as Pebax™. The material of the liner layer 1414 can be stiffer than the polymer of the first stiffness 1408. The material of the liner layer can have a higher value on the hardness/durometer scale than the second polymer of the first stiffness.

The second polymer can continue to extend proximally along the insert catheter 1400, but with increasing stiffness values in a proximal direction. In some implementations, the second polymer of a second stiffness 1410 can extend immediately proximal to the second polymer of the first stiffness 1408. The second stiffness is greater than the first stiffness. The polymer of the second stiffness can have a higher value on the hardness/durometer scale than the polymer of the first stiffness. In some embodiment, the second polymer of the second stiffness can be the same material as the inner liner layer 1414. The length of the second polymer of the second stiffness 1410 can vary depending on a shape of the distal tip, ranging from about 6.5 cm to about 15.5 cm. The insert catheters can have pre-shaped distal tips, which include one or more curves that are pre-set so that the catheter tip assumes the shape rather than being straight when there is no net external force on the catheter tip. The second polymer of the second stiffness 1410 can extend the entire pre-shaped tip.

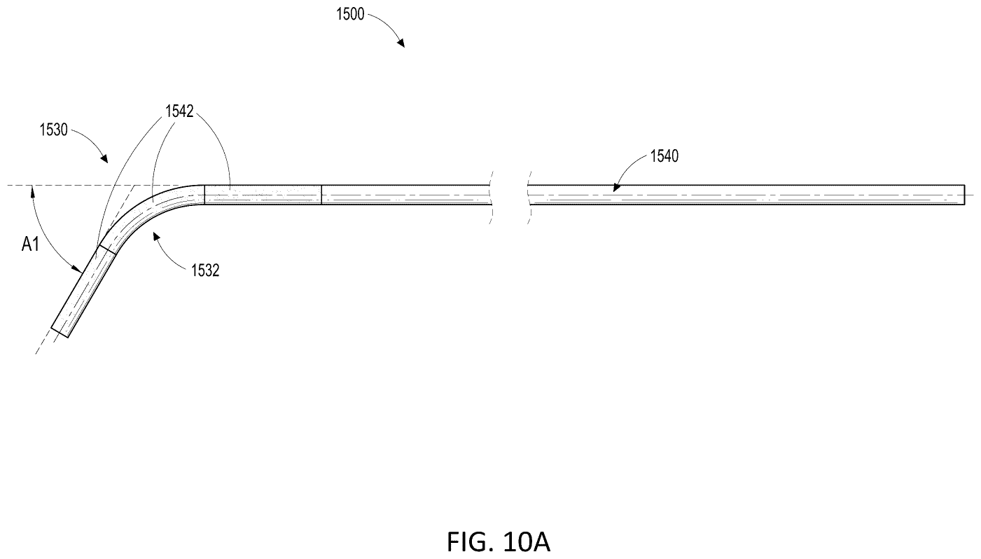

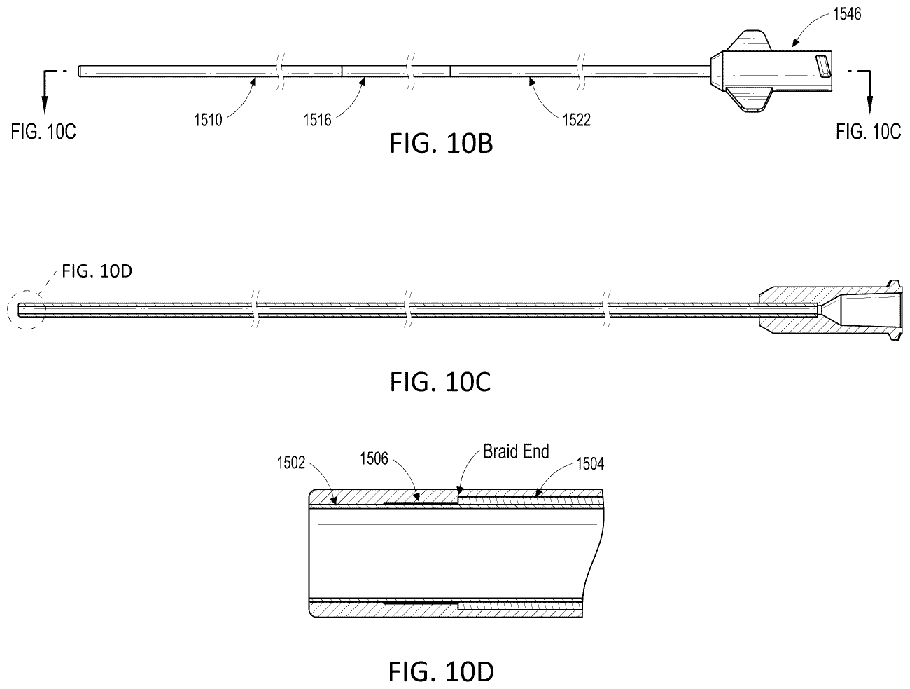

FIGS. 10A-12D illustrate three different types of pre-shaped distal tips that differ in shapes and/or lengths, which will be describe further below in greater detail. The pre-shaped tip can be stiffer than a straight catheter, thereby improving a torque response of the catheter. As will also be described in greater details below with reference to FIGS. 10A-12D, in some embodiments, a distal portion of the braid can be annealed. In some embodiments, the length of the annealed bread may depend on the pre-shaped tip. In some embodiments, the annealed portion may extend through at least a distal tip corresponding to the soft distal tip 1402 as shown in FIGS. 9A-9D as disclosed herein elsewhere, for example, extending through the distal tip corresponding to the soft distal tip 1402, and over the radiopaque marker 1406 and a distal segment of the braid 1404. In some embodiments, the annealed portion may extend through only a distal portion corresponding to the soft distal tip 1402 (or corresponding to the soft distal 1402, the radiopaque marker 1406 and the distal segment of the braid 1404) as shown in FIGS. 9A-9D as disclosed herein elsewhere. That is, the annealing terminates after a distal portion corresponding to the soft distal tip 1402 (or corresponding to the soft distal 1402, the radiopaque marker 1406 and the distal segment of the braid 1404) in those embodiments. Said differently, instead of including a first polymer, the distal tip 1402 in FIGS. 9A-9D is annealed (e.g., only the distal tip 1402 of the braid is annealed); or the distal tip 1402, the radiopaque marker 1406 and the distal segment of the braid 1404 (e.g., only the distal tip 1402, the radiopaque marker 1406 and the distal segment of the braid 1404) are annealed. In the embodiments with an annealed distal portion of the braid, the braid may not include a first polymer (e.g., PET) in its distal portion. Annealing the distal portion of the braid can terminate the braid layer and/or improve shape retention of the catheter tip. Alternatively, as shown in FIGS. 9A-9D, the 32-carrier straight braid 1404 may not be annealed, but can have a distal portion of the braid 1404 be contained in the first polymer (e.g., PET), which forms at least partially the soft distal tip 1402.

The second polymer of the second stiffness 1410 can be doped with an additive for radiopacity. Examples of radiopaque additives include tungsten (W), barium sulphate, etc. W, being denser than barium sulphate or other additives commonly used, can provide greater visibility. The second polymer of the second stiffness 1410 can be doped with W. The additive can allow a physician to see the entire pre-shaped tip. The radiopaque addition (e.g., W) combined with the radiopaque marker 1406 on the tip can improve visibility of the catheter tip during the procedure. In some implementations, the amount of W added can be about 30% to about 50%. The second polymer of the first stiffness 1408, and other polymer portions disclosed herein can include barium sulphate (for example, about 30% to about 50%, or about 38%, or about 39%, or about 40%) for additional marking.

Immediately proximal to the second polymer of the second stiffness 1410, the catheter 1400 can include second polymers of increasing stiffness compared to the second stiffness. For example, immediately proximal to the second polymer of the second stiffness 1410, the catheter 1400 can include the second polymer of a third stiffness 1416; immediately proximal to the second polymer of the third stiffness 1416, the catheter 1400 can include the second polymer of a fourth stiffness 1418. The third stiffness can be greater than the second stiffness and the fourth stiffness can be greater than the third stiffness. In some implementations, the polymer of the third stiffness can have a greater value on the hardness/durometer scale than the polymer of the second stiffness; and the polymer of the fourth stiffness can have a greater value on the hardness/durometer scale than the polymer of the thirdness stiffness. In some implementations, the second polymer of the third stiffness 1416 can have a length of about 9.72 mm to about 10.23 mm, or about 10 mm. In some implementations, the second polymer of the fourth stiffness 1418 can have a length of about 19.8 mm to about 20.3 mm, or about 20 mm. The second polymer of the third stiffness 1416 and the second polymer of the fourth stiffness 1418 can each be doped with barium sulphate, similar to the second polymer of the first stiffness 1408.

Immediately proximal to the second polymer of the fourth stiffness 1418, the catheter can include an outer jacket wall 1420 for a remainder of the proximal polymer portion of the catheter. The outer jacket wall 1420 can be made of a third polymer, which may be different from the polymer segments 1408, 1410, 1416, 1418 proximal to the outer jacket wall 1420. In some implementations, the outer jacket wall 1420 can be made of thermoplastic polyamide. In some implementations, the outer jacket wall 1420 can include a lubricant to improve lubricity and reduce wear and friction, and/or to improve tracking at the distal end of the outer jacket wall 1420. For example, the lubricant can include an ultra-high molecular weight silicone (such as EverGlide®).

In some implementations, the catheter 1400 may not include the second polymer of the third and/or fourth stiffness such that the outer jacket wall 1420 is immediately proximal to the polymer of the second stiffness 1410. In some implementations, the catheter 1400 may include more segments of polymer of increasing stiffness than the third and/or fourth stiffness distal to the outer jacket wall 1420. In some implementations, different polymer segments of different stiffness values may not have all have the same polymer material. The polymer segments distal to the outer jacket wall 1420 may have a durometer value between 40D and 72D. The interface between the polymer segments distal to the outer jacket wall 1420 and the outer jacket wall 1420 can include an angled or tapered cut to improve transition between the two different polymers.

The length of a braided polymer shaft from the distal end of the catheter 1400 to a proximal end of the outer jacket wall 1420 can be about 20 cm to about 100 cm, or about 20 cm to about 80 cm, or about 20 cm to about 60 cm, or about 30 cm to about 40 cm, or about 35 cm. Immediately proximal to the outer jacket wall 1420, the insert catheter 1400 can additionally include a hypotube 1422 as shown in FIGS. 9A, 9B, 9D and 9E. In other words, the insert catheter 1400 can include a distal portion formed by a braided polymer shaft and a proximal portion formed by a hypotube with braid over the hypotube. The hypotube 1422 can be formed by Nitinol, stainless steel, or otherwise. In a preferred implementation, the hypotube 1422 can be formed by Nitinol. The hypotube 1422 can have a length (DIM B in FIG. 9A) of about 120 cm to about 150 cm, or about 125 cm to about 145 cm, or about 125 cm, or about 142 cm. The hypotube 1422 and the braid 1404 can be covered by a polymer outer jacket 1424.

The braid 1404 can include a transition zone 1403 such that the braid 1404 continues from the distal polymer portions over to extend along the hypotube 1422. As shown in FIG. 9D, the transition zone 1403 allows the braid 1404 to be radially inward of the polymers of various stiffness values and the outer jacket wall 1420, and be radially outward of the hypotube 1422. As also shown in FIGS. 9A and 9D, a proximal portion 1426 of the outer jacket wall 1420 can overlap with the hypotube 1422. The proximal portion 1426 can have a length of about 0.96 mm or about 1.04 mm, or about 1 mm. The overlapping proximal portion 1426 can ensure a more smooth transition from the distal polymer portion to the proximal hypotube portion. Moreover, the 32-carrier, straight braid design can further ensure a smooth transition between the distal polymer portion and the proximal hypotube portion. The 32-carrier, straight braid design can provide more wires surrounding the hypotube 1422, and/or have a tighter braid angle, which can provide greater support to the hypotube 1422. Even if there is failure in the hypotube 1422 during the medical procedure, the 32-carrier, straight braid can hold the hypotube 1422 to allow the insert catheter to be pull out of the blood vessel.

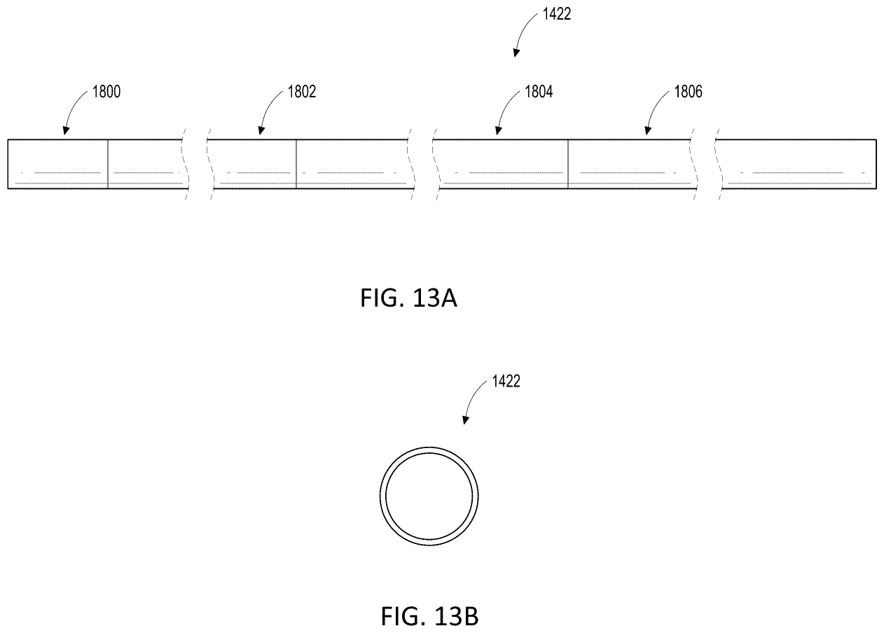

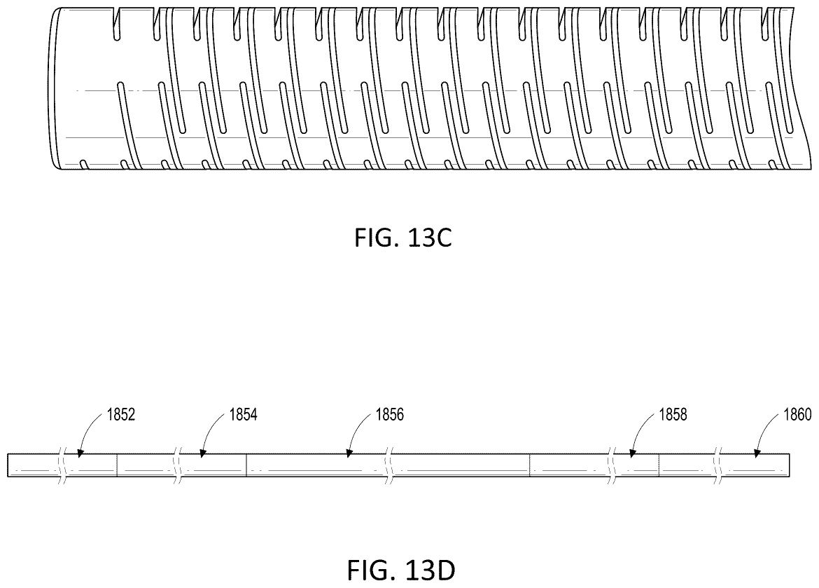

FIGS. 13A and 13B illustrate an example hypotube 1422. The hypotube 1422 can have a wall thickness of about 0.051 mm to about 0.102 mm, or about 0.076 mm. The hypotube can be made of nitinol. The nitinol hypotube 1422 not only can improve column strength of the insert catheter 1400, but also can be advantageous for retaining the torque response of the insert catheter 1400. The insert catheter 1400 may be used for robotic implementations and/or manual implementation. The insert catheter to be driven by a robotic system can have a greater length than a catheter driven manually. For example, the insert catheters as shown in FIGS. 9A-12D, include the catheter 1400, can have a length (DIM A in FIG. 9A) of 150 cm to 190 cm, or 140 cm to 180 cm, or 155 cm to 165 cm, or 175 cm to 190 cm, or about 160 cm, or about 177 cm, or about 187 cm. Due to packaging length constraints, the insert catheters may be packaged bent. A catheter may be prone to taking the bent shape during packaging, which can compromise the torque performance of the insert catheter. It is beneficial that the insert catheters do not take any bent shape when packaged because the torque response of the insert catheters affects the ability of the insert catheters in selecting the appropriate ostium. Due to the shape memory or superelastic property of the Nitinol, the hypotube 1422 can ensure that the insert catheter 1400 restores the straightness and torque performance when removed from a bent packing configuration. The hypotube 1422 can also reduce buckling of the catheter stack (as described elsewhere in the present disclosure, for example, with reference to FIGS. 6 and 7) in the ascending aorta, iliac, and/or outside of the patient's body.

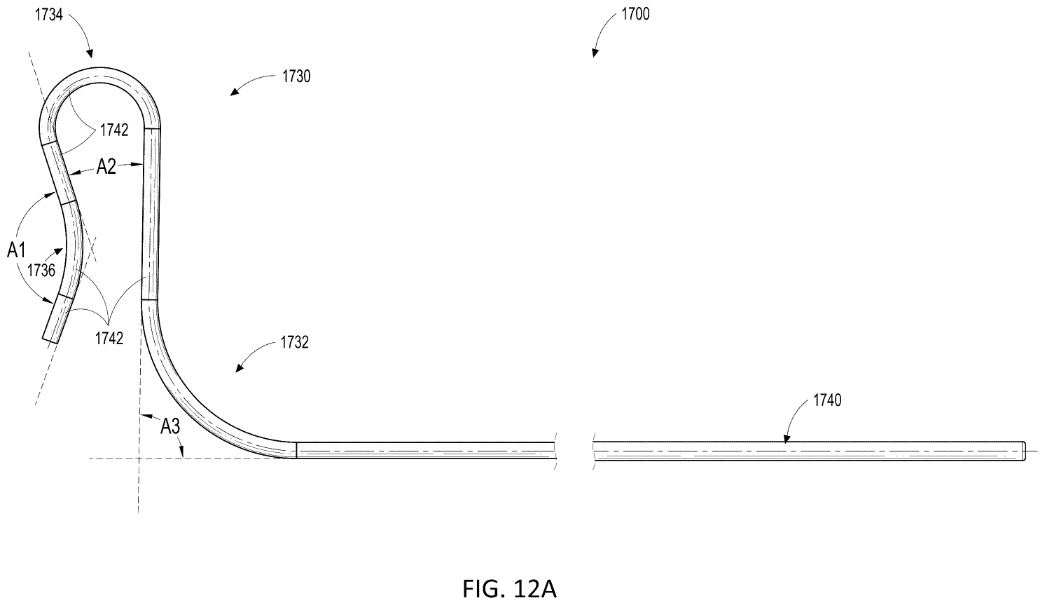

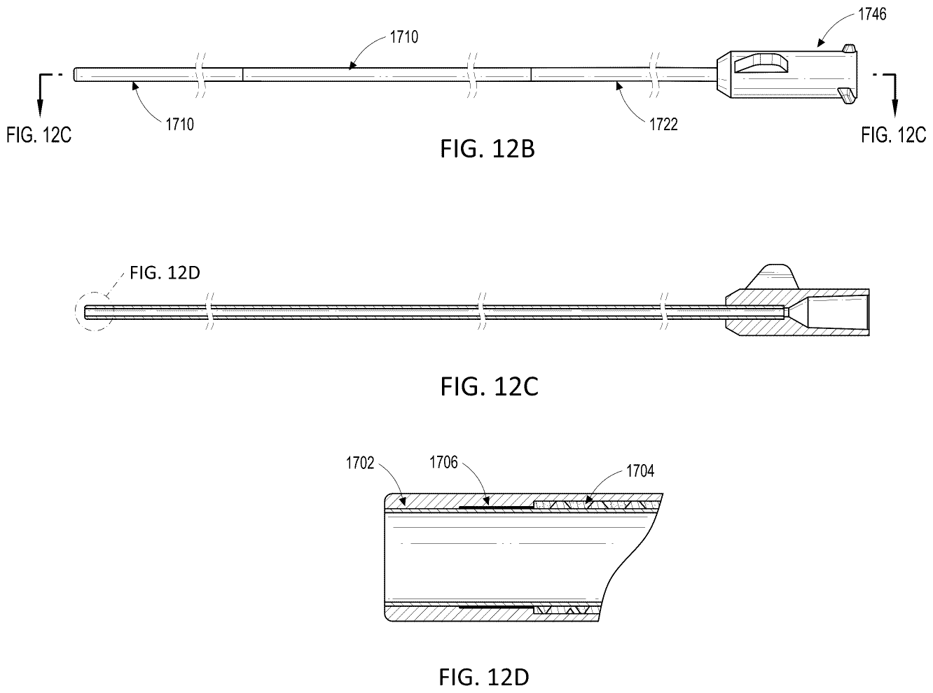

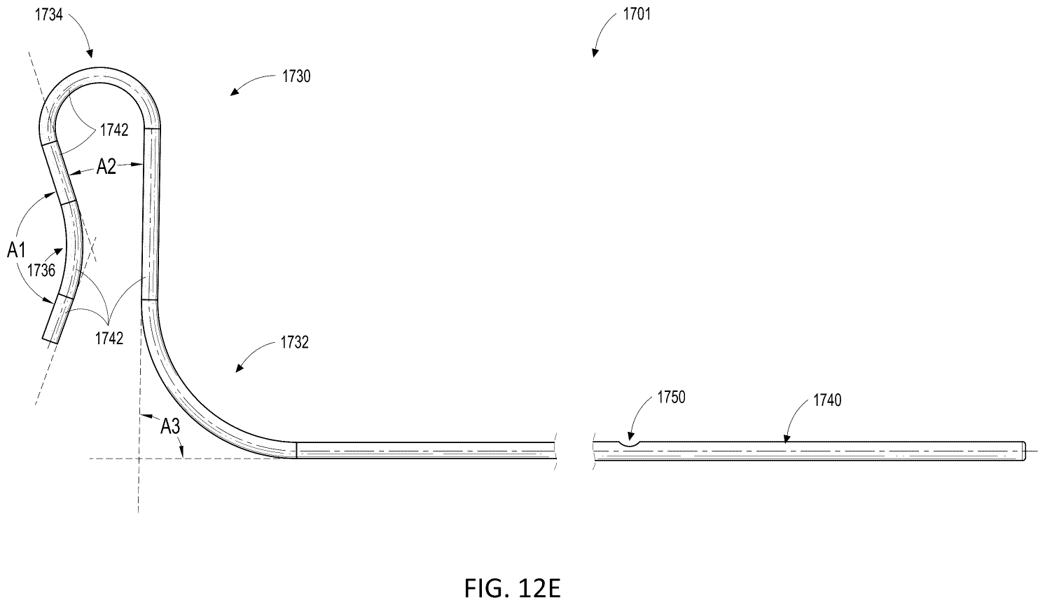

In some implementations, any of the insert catheters disclosed herein can be rapid exchange catheters. Rapid exchange catheters can include a proximal guidewire exit port that is more distal than the proximal end of the catheter. In some implementations, the proximal guidewire exit port can be located on an inner (for example, concave) side of the pre-shaped tip. An example rapid exchange catheter 1701 is shown in FIG. 12E. The catheter 1701 can incorporate any of the features of the catheter 1700, except the difference disclosed herein. The proximal guidewire exit port 1750 is located on a same side as a concave side of the first curve 1732. In other implementations, the proximal guidewire port can be located elsewhere around a circumference of the catheter. Other example insert catheters, including other insert catheters of different pre-shaped tips, can incorporate any features of the rapid exchange catheter 1701.

In some implementations, the proximal guidewire exit port can be located about 20 cm to about 100 cm, or about 20 cm to about 80 cm, or about 20 cm to about 60 cm, or about 30 cm to about 40 cm, or about 35 cm from the distal end of the insert catheter 1400. An additional advantage of the hypotube 1422 forming a proximal portion of the insert catheter 1400 is that the hypotube 1422 can be located adjacent to the proximal guidewire exit port on a proximal side of the proximal guidewire exit port, which can provide reinforcement to the proximal guidewire exit port.

The hypotube 1422 can be laser cut. In some embodiments, for shape retention concerns, it may not be desirable for the laser cut at the proximal end of the hypotube 1422 to include a flexible pattern, which can deform the polymer on the distal braided polymer shaft. Rather, a less complex laser cut pattern can be used. Further, the braid 1404 over the hypotube 1422 can enhance the stability of the braid-to-braid transition from the polymer shaft to the hypotube and improve the manageability of the more rigid hypotube for handling, as described elsewhere in the present disclosure. Different portions of the hypotube 1422 may have different laser cut patterns. For example, as shown in FIG. 13A, a first (distal most) portion 1800 of the hypotube 1422 can have a first laser cut pattern; a second portion 1802 of the hypotube, proximal of the distal most portion 1800, can have a second pattern; a third portion 1804, proximal of the second portion 1802, can have a third pattern; and a fourth (proximal most) portion 1806 can have a fourth pattern. The different laser cut patterns can have different cut per rotation (CPR), uncut degree, pitch, and/or Kerf (the width of material that is removed during the cutting process) values. In some implementations, the laser cut pattern on the hypotube 1422 can have increasing CPRs from the first pattern to the fourth pattern. In some implementation, the laser cut pattern on the hypotube can have increasingly greater uncut degree from the first pattern to the fourth pattern. In some implementations, the laser cut pattern on the hypotube 1422 can have increasing pitch from the first pattern to the fourth pattern. In some implementations, the lengths of the first, second, third, and fourth portions, 1800, 1802, 1804, 1806 can increase from the distal end to the proximal end of the hypotube 1422. The transitions from the first pattern to the fourth pattern can ensure a more gradual transition of the cut pattern along the length of the hypotube 1422 from its distal end to its proximal end.

On other embodiments, the hypotube can include a flexible cut pattern at both the proximal and distal ends, while also incorporating any of the features of the hypotube as shown in FIG. 13A and disclosed herein. In some embodiments, the flexible cut pattern at the proximal end of the hypotube may act as a strain relief and/or facilitate insertion and/or removal of the insert catheter from the overall system due to the hub being at the proximal end of the hypotube (e.g., to reduce cracking during removal, to help with less distance between the pucks, etc.).

FIG. 13C illustrates an example flexible cut pattern, which may be used at the proximal end, the distal end, or both the proximal and distal ends of the hypotube. As shown in FIG. 13C, the flexible laser cut pattern may include an interrupted spiral pattern. In some embodiments, the proximal end and the distal end of the hypotube include the same laser cut pattern (e.g., flexible laser cut pattern).

In some embodiments, the flexible cut pattern at the distal and proximal ends may include a lower pitch than a remainder of the hypotube between the proximal and distal ends. In some embodiments, the flexible cut pattern may be longer at the distal end than at the proximal end. The length of the distal end with the lower pitch cut pattern can be between about 5 cm to about 9 cm, or about 6 cm to about 8 cm, or about 6 cm, or about 7.5 cm, or about 7.6 cm, or about 7.7 cm, or any length within a range defined by any of those values. The length of the proximal end with the lower pitch cut pattern can be between about 11.0 cm to about 15.0 cm, or about 12.0 cm to about 14.0 cm, or between about 13.0 cm to about 13.5 cm, or about 13.1 cm, or about 13.2 cm, or about 13.3 cm, or about 13.4 cm, or any length within a range defined by any of those values. Part of the proximal end with the lower pitch cut pattern can be inside the hub (e.g., about 4 cm to about 7 cm, or about 4.5 cm or about 6 cm, or about 5 cm, or about 6.3 cm, or about 6.4 cm, or any length within a range defined by any of those values). The remainder of the proximal end with the lower pitch flexible cut pattern can remain outside the hub to facilitate removal as disclosed herein.

FIG. 13D illustrates a hypotube including the proximal end 1860 and the distal end 1852 with the lower pitch flexible cut pattern. The hypotube can include a middle portion 1856 with a much greater pitch (e.g., about 5 time to about 10 times greater) than the pitch of the cut pattern at the proximal end 1860 and the distal end 1852. The hypotube can further include transition portions 1854, 1858 between the middle portion 1856 and the proximal end 1860 and the distal end 1852, respectively.

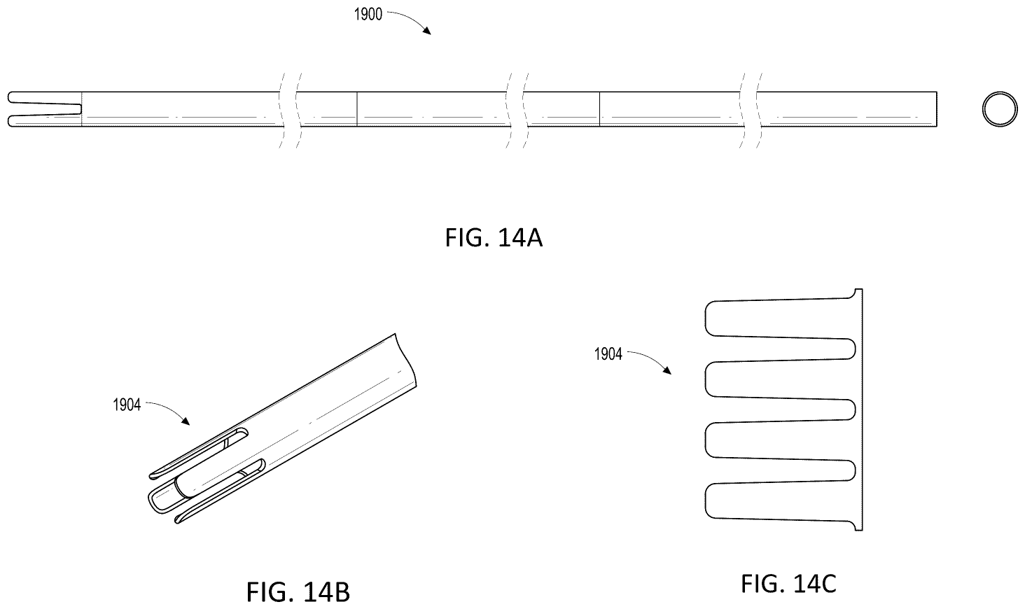

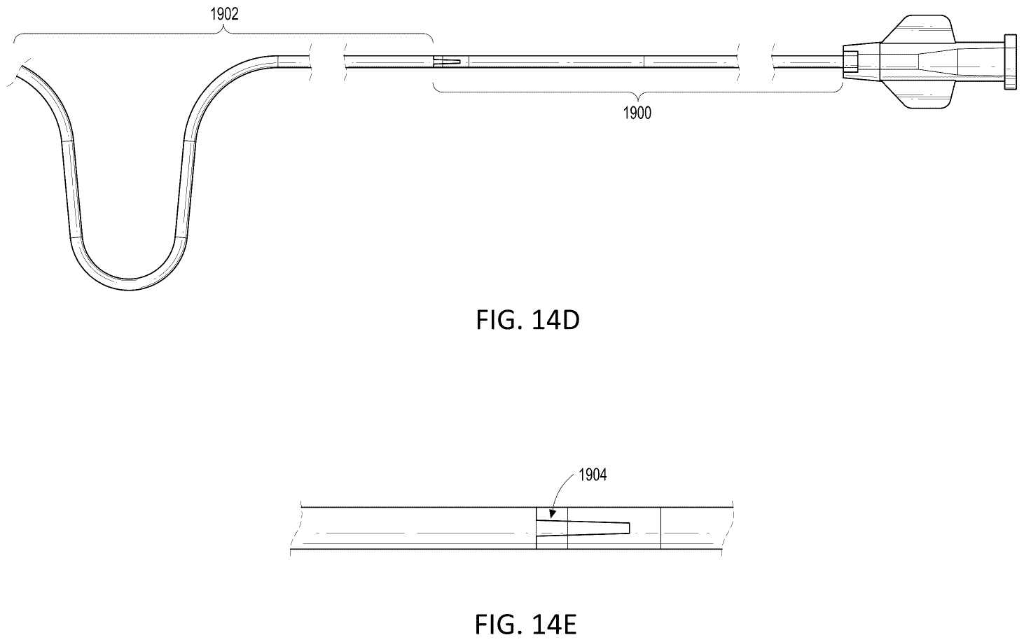

FIGS. 14A-14E illustrate an alternative interface between a hypotube 1900 and the distal polymer shaft 1902. As shown, the distal end of the hypotube 1900 can include finger cut 1904 pattern to overlap the braid of the polymer shaft to avoid an abrupt transition (which can cause failure points, e.g., cracking). Other than the finger cut 1904, the hypotube 1900 can incorporate any of the features of the hypotube 1422.

Turning to FIGS. 10A-10D, the insert catheter 1500 can have any of the features of the catheter 1400 disclosed herein. Description of the insert catheter 1500 will focused on the pre-shaped distal tip 1530. The inserter catheter 1500 can have a pre-shaped distal tip 1530. The pre-shaped distal tip 1530 can include a curve 1532 so that the portion of the tip 1530 distal to the curve 1532 turns away from a longitudinal axis 1540 of the portion of the catheter 1500 proximal to the curve 1532 at an angle A1. The angle A1 can be between about 30° and about 90°. For example, the angle A1 can be between about 40° and about 80°, between about 50° and about 70°, and/or between about 60° and about 65°. The curve 1532 can have a radius of about 8 mm to about 12 mm, or about 9 mm to about 11 mm, or about 10 mm. In some implementations, the insert catheter 1500 can have a pre-shaped tip of a vertebral (VERT or VRT) catheter.

The insert catheter 1500 can include one or more segments 1510, 1516 of polymers with increasing stiffness values from the distal end towards the proximal end in the distal braided polymer shaft and a hypotube 1522 in the proximal portion, with the braid continuing over the hypotube 1522. The segments 1510, 1516 can use the same material as the segments 1410, 1416 of the catheter 1400. At the proximal end, the insert catheter 1500 can be coupled to a hub 1546, which can be any insert catheter hub disclosed herein.

As disclosed elsewhere in the present disclosure, the pre-shaped distal tip 1530 can include a polymer of the second stiffness being doped with W. As shown in FIG. 10D, the pre-shaped distal tip 1530 can further include the soft distal tip 1502, the radiopaque marker 1506, and a distal portion of the braid 1504. As disclosed elsewhere in the present disclosure, the portion of the braid 1504 may optionally be annealed, with the length of the annealed braid depending on the length of the pre-shaped tip 1530, which can be about 2.5 cm to about 3.5 cm, or about 3.0 cm. The annealed braid 1542 is shaded in FIG. 10A.

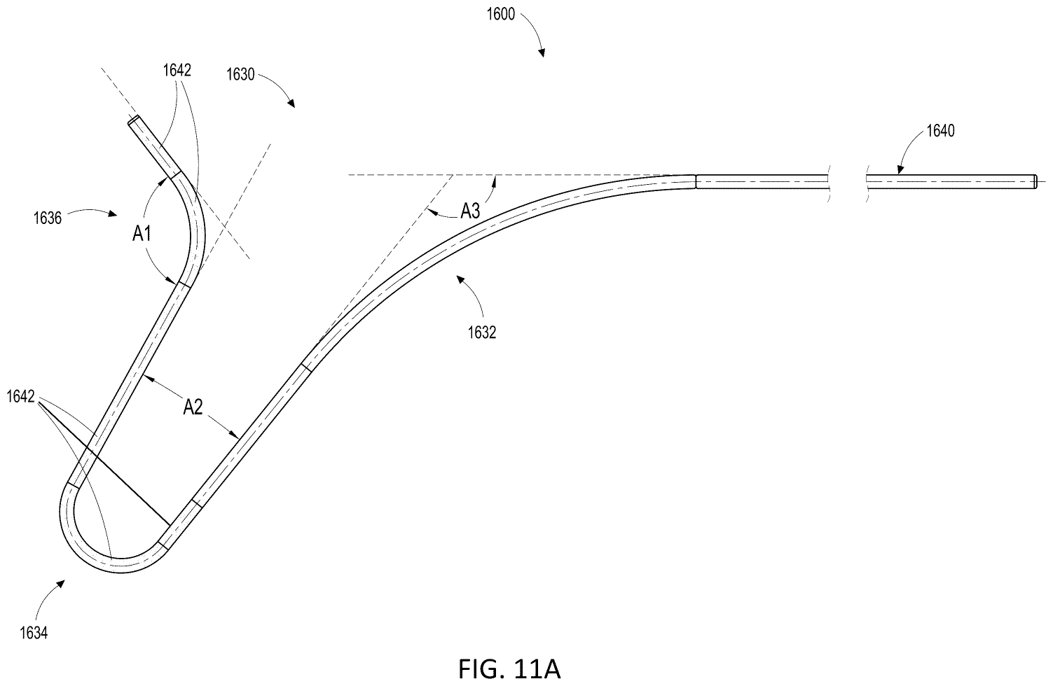

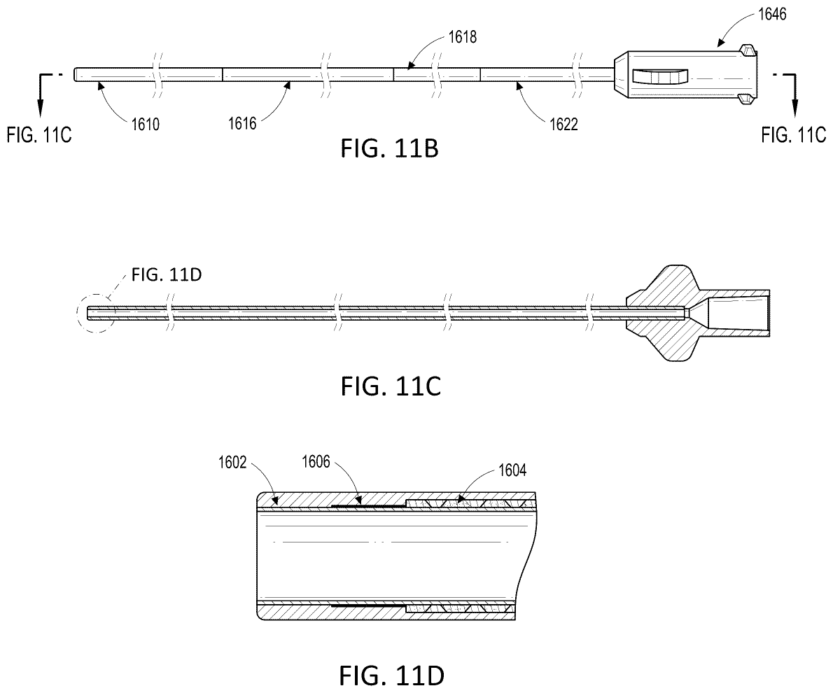

Turning to FIGS. 11A-11D, the insert catheter 1600 can have any of the features of the catheter 1400 disclosed herein. Description of the insert catheter 1600 will focused on the pre-shaped distal tip 1630. The inserter catheter 1600 can have a pre-shaped distal tip 1630. The pre-shaped distal tip 1630 can include a first curve 1632, a second curve 1634, and a third curve 1636. The second curve 1634 can be more distal than the first curve 1632. The third curve 1636 can be a distal most curve. In some implementations, the insert catheter 1600 has the pre-shaped tip of a Simmons catheter.

The portion of the insert catheter 1600 immediately on the proximal side of the first curve 1632 and the portion of the insert catheter 1600 immediately on the distal side of the first curve 1632 (i.e., the proximal most portion of the tip 1630) can establish an angle A3. The angle A3 can be between about 100° and about 160°. For example, the angle A3 can be between about 110° and about 140°, or between about 120° and about 130°. The portion of the insert catheter 1600 on the proximal side of the first curve 1632 can have a longitudinal axis 1640. The portion of the insert catheter 1600 on the distal side of the first curve 1632 can be at an oblique takeoff angle A3 from the longitudinal axis 1640. The first curve 1632 can have a radius of about 60 mm to about 66 mm, or about 61.5 mm to about 64.5 mm, or about 63.0 mm to about 63.5 mm.

The second curve 1634 results in the tip 1630 reversing back toward the longitudinal axis 1640. The portions of the tip 1630 immediately on the proximal and distal sides of the second curve 1634 can establish an angle A2. The angle A2 can be between about 1° and about 50°. For example, the angle A2 can be between about 5° and about 30°, between about 7° and about 20°, and/or between about 10° and about 15°. The second curve 1634 can have a radius of about 4.86 mm to about 8.86 mm, or about 5.86 mm to about 7.86 mm, or about 6.86 mm. The second curve 1634 can allow catheter advancement by pulling rather than pushing, leading to easier and faster catheter placement.

The third curve 1636 turns the tip 1630 away from the first curve 1632. The portions of the tubular body 1405 immediately on the proximal and distal sides of the third curve can establish an angle A1. The angle A1 can be between about 90° and about 140°. For example, the angle A2 can be between about 95° and about 135°, between about 100° and about 130°, and/or between about 110° and about 120°. The third curve 1636 can have a radius of about 10 mm to about 15 mm, or about 11 mm to about 14 mm, or about 12 mm to about 13 mm. The third curve can help in the anchorage at the ostium.

The insert catheter 1600 can include one or more segments 1610, 1616, 1618 of polymers with increasing stiffness values from the distal end towards the proximal end in the distal braid polymer shaft and a hypotube 1622 in the proximal portion, with the braid continuing over the hypotube 1622. The segments 1610, 1616, 1618 can use the same material as the segments 1410, 1416, 1418 of the catheter 1400. At the proximal end, the insert catheter 1600 can be coupled to a hub 1646, which can be any insert catheter hub disclosed herein.

As disclosed elsewhere in the present disclosure, the pre-shaped distal tip 1630 can include a polymer of the second stiffness being doped with W. As shown in FIG. 11D, the pre-shaped distal tip 1630 can further include the soft distal tip 1602, the radiopaque marker 1606, and a distal portion of the braid 1604. As disclosed elsewhere in the present disclosure, the portion of the braid 1604 may be optionally annealed. The length of the annealed braid depends on the length of the pre-shaped tip 1630, which can be about 7.5 cm to about 8.5 cm, or about 8.0 cm. The annealed braid 1642 is shaded in FIG. 11A, including a distal portion of the tip 1630 extending from the second curve 1634 to the distal end of the catheter 1600.

Turning to FIGS. 12A-12D, the insert catheter 1700 can have any of the features of the catheter 1400 disclosed herein. Description of the insert catheter 1700 will focused on the pre-shaped distal tip 1730. The inserter catheter 1700 can have a pre-shaped distal tip 1730. The pre-shaped distal tip 1730 can include a first curve 1732, a second curve 1734, and a third curve 1736. The second curve 1734 can be more distal than the first curve 1732. The third curve 1736 can be a distal most curve. In some implementations, the insert catheter 1700 has the pre-shaped tip of a Vitek (VTK) catheter.

The portion of the insert catheter 1700 immediately on the proximal side of the first curve 1732 and the portion of the tip 1730 immediately on the distal side of the first curve 1632 (i.e., the proximal most portion of the tip 1730) can establish an angle A3. The angle A3 can be between about 40° and about 140°. For example, the angle A3 can be between about 50° and about 130°, between about 60° and about 120°, between about 70° and about 110°, between about 80° and about 100°, and/or between about 85° and about 95°. The portion of the catheter 1700 on the proximal side of the first curve 1700 can define a longitudinal axis 1740. The portion of the tip 1730 immediately on the distal side of the first curve 1732 can be at a takeoff angle A3 from the longitudinal axis 1740. The first curve 1732 can have a radius of about 12 mm to about 18 mm, or about 13 mm to about 17 mm, or about 14 mm to about 15 mm.

The second curve 1734 results in the tip 1730 reversing back (or U-turning) toward the longitudinal axis 1740 and converging toward the portion of the tip 1730 between the first curve 1732 and the second curve 1734. The portions of the tip 1730 immediately on the proximal and distal sides of the second curve 1634 can establish an angle A2. The angle A2 can be between about 1° and about 70°. For example, the angle A2 can be between about 5° and about 60°, between about 10° and about 40°, and/or between about 15° and about 20°. The second curve 1734 can have a smaller radius than the first curve 1732 or the second curve 1736, for example, about 5 mm to about 9 mm, or about 6 mm to about 8 mm, or about 6.5 mm to about 7 mm.

The third curve 1736 again turns the distal most portion of the tip 1730 away from the portion of the tip 1730 between the first curve 1732 and the second curve 1734 so that the distal end of the catheter 1700 points distally. The portions of the tip 1730 immediately on the proximal and distal sides of the third curve 1736 can establish an angle A1. The angle A1 can be between about 100° and about 200°. For example, the angle A1 can be between about 110° and about 190°, between about 120° and about 180°, between about 130° and about 170°, and/or between about 140° and about 150°. The third curve 1736 can have a radius of about 12 mm to about 18 mm, or about 13 mm to about 17 mm, or about 14 mm to about 15 mm.

The insert catheter 1700 can include one or more segments 1710 of polymers with a predetermined stiffness value in the distal braid polymer shaft and a hypotube 1722 in the proximal portion, with the braid continuing over the hypotube 1722. The segments 1710 can use the same material as the segment 1410 of the catheter 1400. At the proximal end, the insert catheter 1700 can be coupled to a hub 1746, which can be any insert catheter hub disclosed herein.

As disclosed elsewhere in the present disclosure, the pre-shaped distal tip 1730 can include a polymer of the second stiffness being doped with W. As shown in FIG. 12D, the pre-shaped distal tip 1730 can further include the soft distal tip 1702, the radiopaque marker 1706, and a distal portion of the braid 1704. As disclosed elsewhere in the present disclosure, the portion of the braid 1704 can optionally be annealed. The length of the annealed braid depends on the length of the pre-shaped tip 1730, which can be about 5.5 cm to about 6.5 cm, or about 6.0 cm. The annealed braid 1742 is shaded in FIG. 12A, including a distal portion of the tip 1730 extending from the second curve 1734 to the distal end of the catheter 1700.

In certain embodiments, the insert catheters described herein may be designed to have an appropriate flexibility profile to navigate the tortuosity of the anatomy (e.g., an artery) and also to provide a physician with sufficient support and force along the length of the distal end of the catheter so as to advance the distal tip of the catheter to a target location.

In some embodiments, interventional device stiffness or flexibility or interventional device stack stiffness or flexibility can be determined using the cantilever beam test. The cantilever beam test can be done along the length of an interventional device or interventional device stack to characterize the local segment stiffness and the rate of stiffness change. For example, in certain embodiments, the peak load may be measured at one or more increments along an interventional device or interventional device stack. In some embodiments, the flexibility of an interventional device or interventional device stack may be tested at 6 mm increments along a length of at least a portion of the interventional device or interventional device stack. In some embodiments, an interventional device of interventional device stack can be secured at a 5 mm distance from a target location to be measured. This is the gage length (e.g., 5 mm) used by the cantilever beam test. A force can then be applied to cause a 4 mm displacement at the target location of the interventional device or interventional device stack, wherein the peak load is measured within the 4 mm displacement. The amount of force applied is measured at each sampling location (e.g., every 6 mm) to create a flexibility or stiffness profile for the interventional device or interventional device stack. The flexibility or stiffness profile can illustrate the peak load (gram-force or gF) experienced at each sampling location (e.g., every 6 mm) along the length of the interventional device or interventional device stack.

Additionally or alternatively, the interventional device (e.g., the insert catheters disclosed herein) stiffness can be determined using a three-point bending test, in which the portion of the catheter to be test is supported at both ends and loaded centrally. In some embodiments, three-point bending test may be performed per the ASTM D790 standard, the ISO 7438 standard, and/or the ISO 178 standard. In some embodiments, the pre-shaped distal tip of the insert catheter (e.g., the section with the second polymer of the second stiffness 1410 as disclosed herein elsewhere) may have a stiffness of about 125-140 gF; the transition portion immediately proximal to the pre-shaped distal tip (e.g., the section with the second polymer of the third stiffness 1416 and the second polymer of the fourth stiffness 1418 as disclosed herein elsewhere) may have a stiffness of about 275 gF to 295 gF. On the proximal side of the second polymer of the fourth stiffness 1418, the section with the outer jacket wall 1420 as disclosed herein elsewhere may have a stiffness of about 630 to 645 gF. Proximal to the braided shaft, the hypotube flexible sections (e.g., at the proximal and distal ends) may have a stiffness of about 950 to 965 gF; and the hypotube stiff sections (e.g., between the proximal and distal ends with the flexible cut pattern) may have a stiffness of about 1845 to 1860 gF. In the embodiments where the proximal end of the hypotube does not include a flexible cut pattern, the proximal end of the hypotube may have a stiffness of about 1845 to 1860 gF.

Example Sensors and/or Markers

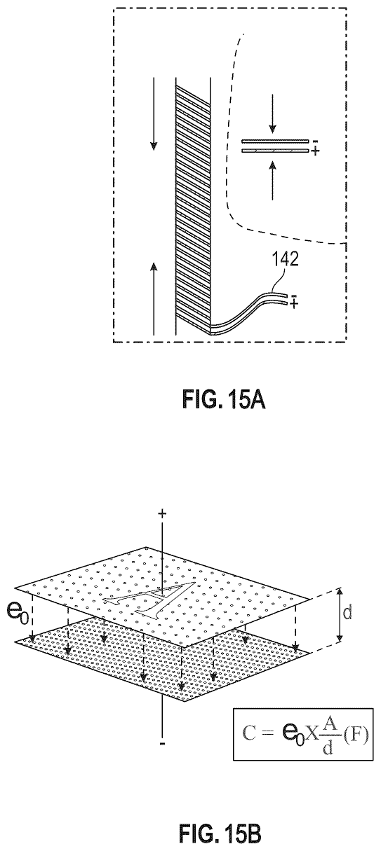

Any of a variety of sensors may be provided on any of the catheters, hubs, carriages, or table, depending upon the desired data. For example, in some implementations, it may be advantageous to measure axial tension or compression force applied to the catheter such as along a force sensing zone. The distal end of the catheter can be built with a similar construction as illustrated in FIG. 6, with a helical coil distal section. Alternatively, instead of using a single helical coil of nitinol wire, a first conductor 140 and second conductor 142 can be wrapped into intertwined helical coils and electrically isolated from each other such as by the plastic/resin of the tubular body, such as shown in FIG. 15A. Each coil can be in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 15B. The capacitance is inversely proportional to the distance between wires. The only variable that would be changing would be d, the distance between the plates. If an axial compressive force is applied to the catheter, the wires (e.g., conductor 140 and conductor 142) can move closer together, thus increasing the capacitance. If an axial tensile force is applied, the wires can get further apart, decreasing the capacitance. This capacitance can be measured at the proximal end of the catheter, giving a measurement of the force at the helical capacitor. Although referred to as a capacitor, this sensor is measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 centimeters of the catheter body to sense forces experienced at the distal end. At least a second capacitor may be provided within the proximal most 5 or 10 or 20 centimeters of the catheter body, to sense forces experienced at the proximal end of the catheter.

It may also be advantageous to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub can move a small amount relative to the carriage. In robotics, this is called a series elastic actuator. This property can be used to measure the force applied from the carriage to the hub. To measure the force, the relative distance between the hub and the carriage is determined and characterize some effective spring constant between the two components.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magneto-resistive sensor, or another type of magnetic field sensor. Generally, multiple sensors may be used to increase the reliability of the measurement. This reduces noise and reduces interference from external magnetic fields.

Other non-contact distance sensors can also be used. These may include optical sensors, inductance sensors, and capacitance sensors. Optical sensors can be configured in a manner that avoids accumulation of blood or other fluid in the interface between the hubs carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory, or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers can sense the shape of catheters and guidewires. By using multiple force sensing fibers that are a known distance from each other, the shape along the length of the catheter/guidewire can be determined.

A strain gauge may be integrated into the body of the catheter or guidewire to measure force or torque. In an example, the string gauge is a resistive strain gauge. In some embodiments, the strain gauge is incorporated in the distal tip of the catheter, or incorporated in the proximal end of the catheter, and/or incorporated in the proximal end of the catheter and the proximal end of the catheter. In some embodiments, a strain gauge can be deposited on a wall of the catheter via thin film deposition technologies.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table can include a linear array of corresponding magnetic sensors along the entire length of the table. A processor can be configured to determine the location of the magnet along the length of the linear sensor array and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Distal tip position or movement or lack thereof may be compared with commanded or actual proximal catheter or guidewire movement at the hub to detect a loss of relative motion. The loss of relative motion may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique may only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy. Machine vision can also be used to analyze catheter straightness relative to build up tension. As tension builds up in the catheter due to navigating anatomical tortuosity, the catheter can begin to bow and buckle in a sinusoidal manner thus becoming less straight. This tension build can lead to more severe prolapse if built up enough. Measuring the loss of straightness can provide a signal to stop before prolapse can happen. This can be particularly helpful when the area of prolapse is outside of the current x-ray view.

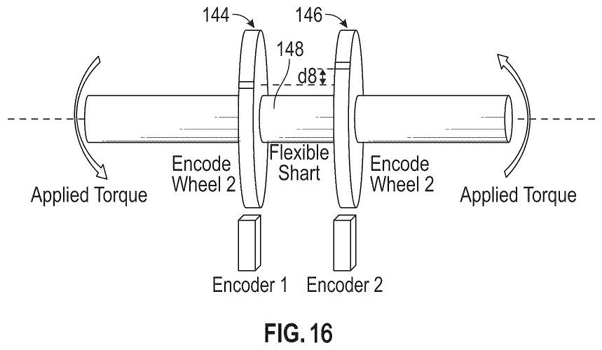

Proximal torque applied to the catheter or guidewire shaft may be determined using a dual encoder torque sensor. Referring to FIG. 16, a first encoder 144 and a second encoder 146 may be spaced axially apart along the shaft 148, for measuring the difference in angle over a length of flexible catheter/tube. The difference in angle is interpolated as a torque since the catheter/tube has a known torsional stiffness. As torque is applied to the shaft, the slightly flexible portion of the shaft can twist. The difference between the angles measured by the encoders (dθ) can be used to calculate the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In some embodiments, bubbles can be dislodged from the catheter wall via a beam ultrasonic energy from an ultrasound transducer. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow.

It may additionally be advantageous for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, filed on Nov. 16, 2021, the entirety of which is hereby incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

### Example Interventional Devices Control Mechanism

In some embodiments, a control mechanism (e.g., a controller, such as a handheld controller) may be used for manipulating interventional devices driven by (or otherwise associated with) respective hubs. For example, each hub may be manipulated and/or otherwise moved using at least one control installed in control mechanism. Each control may be adapted to move a unique hub and associated interventional device during an interventional procedure. Alternatively, a single control may be adapted to control multiple hubs and associated interventional devices. In some embodiments, a control may be adapted to cause translation and/or rotation of one or more hubs and associated interventional devices. In some embodiments, a single control may be adapted to cause both translation and rotation. In other embodiments, separate controls may cause translation and rotation of a single hub and associated interventional device or of multiple hubs and associated interventional devices.

A control mechanism may be positioned on or near to a patient support table having a set of hubs and catheters/interventional devices. In some implementations, the control mechanism may be positioned remote from the support table such as behind a radiation shield or in a different room or different geographical location in a telemedicine implementation.

In some implementations, other control operations beyond translational movement and rotational movement may be carried out using controls as described herein. For example, one or more controls may be configured to drive a shape change and/or stiffness change of a corresponding interventional device. The controls may be toggled between different operating modes. For example, the controls may be toggled between movement driven by acceleration and velocity to movement that reflects actual linear displacement or rotation.

In some implementations, the control mechanism may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

In some implementations, the control mechanism may include haptic components to provide haptic feedback to a user operating the controls. For example, if the control 2202 is triggering movement of a catheter and the catheter detects a large force at the tip, the control 2202 may generate haptic feedback to indicate to the user to stop or reverse a performed movement. In some implementations, haptic feedback may be generated at the control to indicate to the user to slow or speed a movement using the control. In some implementations, haptics may provide feedback on a large torsional strain buildup that might precede an abrupt rotation, or a large axial force buildup that may be a prelude to buckling of the catheter.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. A real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

Additional details regarding a controller for a robotic surgical system can be found in U.S. patent application Ser. No. 18/784,630, entitled SYSTEM FOR REMOTE MEDICAL PROCEDURE, filed Jul. 25, 2024, U.S. patent application Ser. No. 18/525,267, entitled METHOD FOR ROBOTICALLY CONTROLLING SUBSETS OF INTERVENTIONAL DEVICE ASSEMBLY, filed Nov. 30, 2023, and U.S. patent application Ser. No. 19/228,468, titled METHOD FOR ROBOTICALLY CONTROLLING INTERVENTIONAL DEVICE ASSEMBLY, filed Jun. 4, 2025, the entirety of each of which is hereby incorporated by reference herein.

### Example Method of Use of the Catheter Assembly

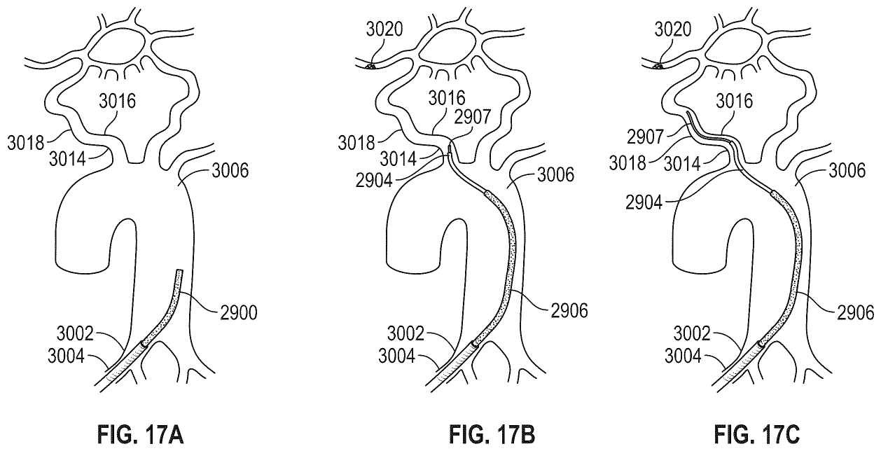

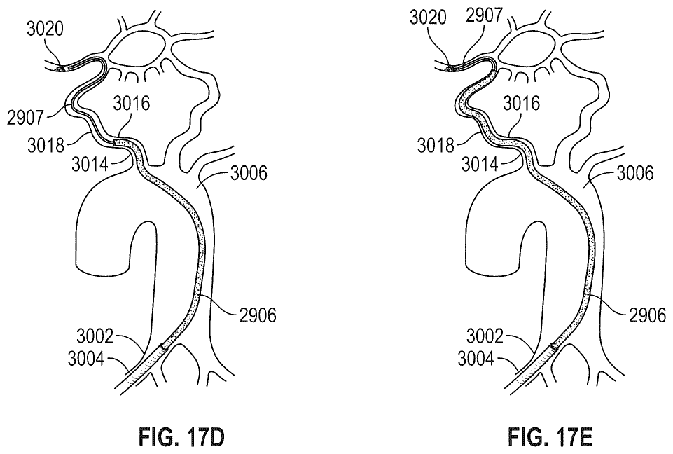

FIGS. 17A-17E depict an example sequence of steps of introducing a multi-catheter assembly configured to achieve access all the way to the clot, either manually or robotically. FIGS. 17A-17E may be described using the interventional device assembly of FIG. 6. Other combinations of catheters may be substituted for the interventional device assembly, as will be appreciated by those of skill in the art in view of the disclosure herein.

Referring to FIG. 17A, the three-catheter interventional device assembly 2900 is shown driven through an introducer sheath 3002, up through the iliac artery 3004 and into the descending aorta. Next, the insert catheter 2902 (which can be, for example, any of the insert catheters described with reference to FIGS. 9A-14E), the procedure catheter 2904 (e.g., 0.071 inch) and the guide catheter 2906 (e.g., 0.088 inch) are tracked up to the aortic arch 3006, as shown in FIG. 17B. Here, the distal end of the guide catheter 2906 may be parked below the aortic arch 3006 and the procedure catheter 2904, insert catheter 2902 (positioned within the procedure catheter 2904 and not visible in FIG. 17B) and a guidewire 2907 can be driven into the ostium (e.g., simultaneously or separately). In some embodiments, the insert catheter 2902 is advanced out of the procedure catheter 2904 and the guide catheter 2906 to engage the ostium first. After the distal end of the insert catheter 2902 is positioned within the desired ostium, the guidewire 2907 can be advanced distally into the ostium to secure (or guide) access, and/or to confirm proper vessel selection and positioning. After the insert catheter 2902 and guidewire 2907 are positioned within the desired ostium, the procedure catheter 2904 and/or guide catheter 2906 can be advanced into the ostium (and, in some embodiments, beyond), while using the support of the insert catheter 2902 and/or guidewire 2907 to maneuver through the aorta and into the ostium. In the embodiment shown in FIG. 17B, the procedure catheter 2904 has been advanced into the ostium while the guide catheter 2906 has remained parked below the aortic arch 3006.

Referring to FIG. 17C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 17D, the guide catheter 2906 and the procedure catheter 2904 (positioned within the guide catheter 2906 and not visible in FIG. 17D) are both advanced (e.g., simultaneously or sequentially) over the guidewire 2907 and over the insert or access catheter 2902 (positioned within the procedure catheter 2904 and not visible in FIG. 17D) while the access catheter 2902 remains at the ostium for support. The guidewire 2907 may be further advanced past the petrous segment 3018 to the site of the clot 3020, such as the M1 segment.

Referring to FIG. 17E, the guide catheter 2906 and the procedure catheter 2904 (positioned within the guide catheter 2906 and not visible in FIG. 17D) are advanced (e.g., simultaneously or sequentially) to position the distal tip of the procedure catheter 2904 at the procedure site, for example on the face of the clot 3020. The guidewire 2907 and insert catheter 2902 (positioned within the procedure catheter 2904 and not visible in FIG. 17E) are removed, and aspiration of the clot 3020 commences through the procedure catheter 2904. That is, the guidewire 2907 and the insert catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

In some embodiments, aspiration may be performed through two catheters (e.g., the procedure catheter 2904 and the guide catheter 2906) simultaneously. For example, during a thrombectomy procedure, the clot 3020 may become engaged with or corked at a distal end of the procedure catheter 2904 (or another inner catheter). In such cases, it may be necessary to remove the procedure catheter 2904 (or other inner catheter) from the vasculature of the patient to remove the clot 3020. As the procedure catheter 2904 (or other inner catheter) is retracted from the guide catheter 2906 (or other outer catheter), debris from the stuck clot 3020 can dislodge. In such embodiments, application of vacuum at both the procedure catheter 2904 (or other inner catheter) and the guide catheter 2906 (or other outer catheter) can beneficially prevent the debris from flowing distally into the vasculature of the patient by aspirating the debris and thus reducing the risk of embolization.

In some embodiments, the clot 3020 may become engaged with or corked at the distal end of the guide catheter 2906 (or other outer catheter). Vacuum through the guide catheter 2906 (or other outer catheter) may prevent dislodgement of the clot 3020 from the guide catheter 2906 (or other outer catheter). In some instances, it may not be readily apparent if portions of a clot are engaged with the guide catheter 2906 (or other outer catheter) or the procedure catheter 2904 (or other inner catheter). In such instances, vacuum through both the guide catheter 2906 (or other outer catheter) and the procedure catheter 2904 (or other inner catheter) may prevent debris from flowing distally into the vasculature of the patient by aspirating the debris and thus reducing the risk of embolization.

The catheter assembly 2900 may be used to perform a neurovascular procedure, as described in FIGS. 17A-17E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system and driving the assembly to achieve supra-aortic access. The steps may further include driving a subset of the assembly to a neurovascular site and performing the neurovascular procedure using a subset of the assembly. The subset of the assembly may include the guidewire, the guide catheter, and the procedure catheter.

Each of the guidewire 2907, the insert catheter 2902, the procedure catheter 2904, and the guide catheter 2906 is configured to be adjusted by a respective hub. For example, the guidewire 2907 may include (or be coupled to) a hub installed on one of the tray assemblies described herein (for example, hub 26, FIG. 3A). Similarly, the insert catheter 2902 may include, or be coupled to catheter hub 2910. The procedure catheter 2904 may include, or be coupled to, the procedure catheter hub 2912. The guide catheter 2906 may include, or be coupled to, the guide catheter hub 2914.

In general, coupling of the assembly 2900 may include magnetically coupling a first hub 2909, which is coupled to the guidewire 2907, to a first drive magnet; magnetically coupling a second hub 2910, which is coupled to the insert catheter 2902, to a second drive magnet; magnetically coupling a third hub 2912, which is coupled to the procedure catheter 2904, to a third drive magnet; and magnetically coupling a fourth hub 2914, which is coupled to the guide catheter 2906, to a fourth drive magnet. In various embodiments, there can be one layer of material, or multiple layers of material (that is, one or more layers of material), between each of the first, second, third, and fourth hubs, and their corresponding first, second, third, and fourth drive magnets, such that the magnetic couplings are through the layer(s) of material. In various embodiments, each layer may be flexible, semi-rigid, or rigid. A layer can be a sterile barrier. When the system is configured for use, the first drive magnet, the second drive magnet, the third drive magnet, and the fourth drive magnet are each independently movable, and movably carried by (or on) a drive table, for example, as described with respect to tray assemblies and controls described herein. In some embodiments, the first drive magnet, the second drive magnet, the third drive magnet, and the fourth drive magnet are coupled to their respective catheter hubs through a sterile barrier (e.g., a sterile and fluid barrier). Each of the first, second, third, and fourth drive magnets may be controlled to be independently movable relative to the other drive magnets. Each of the first, second, third, and fourth drive magnets may be incorporated on a drive table having a plurality of drive magnets that are controlled to move along the drive table (for example, along a longitudinal axis of the drive table). In some embodiments, two or more drive magnets can be tethered or otherwise coupled together to move as a unit in response to commands from a single controller. In some examples, the drive magnets can be coupled together to move together (e.g., physically coupled together or configured to move together by a controller). In an example, the first and second drive magnets may be configured to move together along the drive table. In another example, the first, second, third, and fourth drive magnets may be configured to move together. In another example, any two or more of the first, second, third, and fourth drive magnets (and correspondingly, the hubs the first, second, third, and fourth drive magnets are coupled to) may be configured to be moved together along a drive table.

In some implementations, the steps of performing the neurovascular procedure may include driving the assembly in response to movement of hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a magnet (sometimes referred to herein as a “driven magnet”) carried by the respective hub. This provides a magnetic coupling between the drive magnet and driven magnet through the sterile barrier such that the respective hub is moved across the top of the sterile barrier (within the sterile field) in response to movement of the hub adapter which is positioned outside of the sterile field (as described in detail in FIG. 4). Movement of the hub adapter is driven by a drive system carried by the support table in which the guidewire hub 2909, the insert catheter hub 2910, the procedure catheter hub 2912, and the guide catheter hub 2914 are installed upon.

Movement of the catheter assembly 2900 during a procedure may include moving including a portion on the catheter assembly 2900. For example, moving the catheter assembly 2900 during a procedure may include driving a subset of the assembly in response to movement of one or more of the hub adapters along the support table until the subset of the assembly is positioned to perform a neurovascular procedure at a neurovascular treatment site. The subset of the assembly may include the guidewire 2907, the guide catheter 2906, and/or the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 17A-17E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, insert catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an insert catheter hub 2910 configured to adjust axial and rotational movement of an insert catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect the procedure catheter 2904 through a distal deflection zone.

In some embodiments, the guidewire hub 2909 is configured to couple to a guidewire hub adapter by magnetically coupling the guidewire hub to a first drive magnet. The insert catheter hub 2910 is configured to couple to an insert catheter hub adapter by magnetically coupling the insert catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail in FIG. 4 elsewhere in the present disclosure.

In some embodiments, the robotic control system includes a second driven magnet on the insert catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the insert catheter 2902, the procedure catheter 2904, and/or the guide catheter 2906) and/or one or more hubs (e.g., the guidewire hub 2909, the insert catheter hub 2910, the procedure catheter hub 2912, and/or the guide catheter hub 2914) with one or more drive mechanisms.

The interventional devices described herein may be provided individually, or at least some of the interventional devices can be provided in a preassembled (e.g., nested or stacked) configuration, for example, as part of a sterile kit. In an example, the interventional devices may be provided in the form of an interventional device assembly, such as interventional device assembly 2900, in a concentric nested or stacked configuration. If provided individually, each catheter (and in some embodiments, each corresponding catheter hub) can be unpackaged and primed to remove air from its inner lumen, for example, by flushing the catheter (and in some embodiments, each corresponding catheter hub) to remove air by displacing it with a fluid, such as saline, contrast media, or a mixture of saline and contrast media. After priming, the interventional devices can be manually assembled into a stacked configuration so that they are ready for introduction into the body for a surgical procedure, for example, via an introducer sheath.

Assembling the devices into a stacked configuration can include individually inserting interventional devices into one another by order of size. For example, an interventional device having a second largest diameter can be inserted into the lumen of an interventional device having a largest diameter. An interventional device having a third largest diameter can then be inserted into the interventional device having the second largest diameter and so on.

For example, with respect to FIG. 7, assembly can be performed by first inserting a distal end of the catheter 2904 through the hub 2914 and into the catheter 2906. The catheter 2904 can be advanced through the catheter 2906 until the distal tip of the catheter 2904 is flush with or extends beyond the distal tip of the catheter 2906, and/or until the catheter 2904 cannot be inserted any further. Then, the distal end of the insert catheter 2902 can be inserted through the hub 2912 and into the catheter 2904. The catheter 2902 can be advanced through the catheter 2904 until the distal tip of the catheter 2902 is flush with or extends beyond the distal tip of the catheter 2904, and/or until the catheter 2902 cannot be inserted any further. Then, the distal end of the guidewire 2907 can be inserted through the hub 2910 and into the insert catheter 2902. The guidewire 2907 can be advanced through the catheter 2902 until the distal tip of the guidewire 2907 is flush with or extends beyond the distal tip of the catheter 2902, and/or until the guidewire 2907 cannot be inserted any further.

Embodiments in which two or more of the interventional devices are packaged together as a single unit in an assembled (e.g., nested or stacked) configuration may provide efficient unpackaging and preparation prior to use and efficient assembly within a robotic control system. The interventional devices may be pre-mounted to their respective hubs prior to packaging. In certain embodiments, two or three or more interventional devices may be packaged in a fully nested (i.e., fully axially inserted) configuration or nearly fully nested configuration. In a fully nested configuration, each interventional device is inserted as far as possible into an adjacent distal hub and interventional device. Such a fully nested configuration may minimize a total length of the interventional device assembly and minimize the size of the packaging required to house the interventional device assembly.

In some embodiments, the interventional devices may also be sterilized prior to packaging while in the assembled configuration, for example, using ethylene oxide gas. In some embodiments, the interventional devices may be packaged while in the assembled configuration before sterilization with ethylene oxide gas. For interventional devices in a nested or stacked configuration, ethylene oxide gas can be provided in a space between adjacent interventional devices (for example, an annular lumen between an outer diameter of a first interventional device nested within a second interventional device and the inner diameter of the second interventional device) for sterilization. In some embodiments, the interventional device assembly can be packaged in a thermoformed tray and sealed with an HDPE (e.g., Tyvek®) lid. The interventional device assembly can be unpackaged by removal (e.g., opening or peeling off) of the lid by a user in a sterile field. A user in the sterile field can then remove the interventional device assembly and place it on the sterile work surface, for example, of a robotic drive table, as described herein.

Packaging the interventional devices in an assembled configuration and sterilized state can reduce the time associated with unpackaging and assembly of individual interventional devices and facilitate efficient connection to a robotic drive system. Each interventional device and hub combination may further be packaged with a fluidics connection for coupling to a fluid source, or one or more fluid sources, and/or a vacuum source. In some embodiments, each hub, or a hemostasis valve coupled to the hub, may include the fluidics connection.

After the interventional device assembly is unpackaged (e.g., after the interventional device assembly is positioned on the robotic drive table), priming can be performed while the devices are concentrically nested or stacked. This is preferably accomplished in each catheter lumen, for example, the annular lumen between the guide catheter 2906 and the procedure catheter 2904, and in between each of the additional concentric interventional devices in the catheter stack. In certain embodiments, fluid can be introduced in one or more lumens of the catheter stack to prime one or more interventional devices. For example, fluid can be introduced in a lumen between a distal hub and a proximal interventional device. For example, the lumen between the hub 2914 and the catheter 2904. In certain embodiments, priming can be performed while the devices are in the sterile packaging. More typically, priming of one of more of the catheters in the catheter stack can be performed when the catheter assembly has been removed from its packaging.

The fluidics connections of the catheter assembly (for example, the fluidics connection to one or more of the hubs) can be connected to a fluidics system for delivering saline and contrast media to the catheters, and providing aspiration. In some embodiments, one or more of the fluidics connections (e.g., to saline, contrast, or aspiration) may extend from the sterile field to outside the sterile field for connection to the fluidics system. Once connected, the fluidics system can perform a priming sequence to flush each catheter of the interventional device assembly with fluid (e.g., saline, contrast media, or a mixture of saline and contrast media). The priming sequence may also include flushing each corresponding catheter hub with fluid. The fluid may be de-aired or de-gassed by the fluidics system prior to priming. In some embodiments, a vacuum source of the fluidics system can also be used to evacuate air from each catheter while flushing with fluid. In certain embodiments, a tip of the catheter can be placed into a container of fluid, such as saline, contrast media, or a mixture of saline and contrast media, during priming so that the fluid in the container, and not air, is aspirated through the tip of the catheter when the vacuum source is applied. In other embodiments, the tip of the catheter may be blocked (for example, using a plug) so that air is not aspirated from the tip of the catheter when the vacuum source is applied. In certain embodiments, the priming process may be automated such that a user can provide a single command and each catheter (and in some embodiments, each corresponding catheter hub) can be primed, sequentially or simultaneously.

Additional details regarding fluidics systems are disclosed in U.S. patent application Ser. No. 17/879,614, entitled Multi Catheter System With Integrated Fluidics Management, filed Aug. 2, 2022, which is hereby expressly incorporated in its entirety herein by reference.

Fluid resistance within a lumen may be greater when there is a reduction in cross sectional luminal area for flow, for example, when a second interventional device (e.g., a catheter or guidewire) extends within the lumen of a first interventional device. The amount of fluid resistance can be affected by the length of the cross sectional narrowing, for example, due to a depth of axial insertion of the second interventional device within the first interventional device. A second interventional device extending partially through the lumen of a first interventional device will provide a smaller length of cross-sectional narrowing, and accordingly may result in a lower fluid resistance within the lumen of the first catheter, than if the second interventional device were to extend entirely through the lumen of the first interventional device. Thus, fluid resistance can be lowered by at least partially decreasing a depth of axial insertion (i.e., axial overlap) of a second interventional device into the lumen through which fluid is to be injected (e.g., a length of the second interventional device into its concentrically adjacent lumen).

In some embodiments, over certain depths of insertion of a second interventional device within a first interventional device (for example, when the second interventional device is at or near a maximum insertion depth within the first interventional device), the size of the fluid channel between the devices (e.g., the annular lumen between the first interventional device and the second interventional device) can lead to higher than desirable amounts of fluid resistance during a priming procedure. In some embodiments, the depth of insertion of the second interventional device within the first interventional device can be decreased to reduce the pressure needed to prime the catheter and reduce internal interference.

In some embodiments, a catheter in the interventional device assembly can be separated from the other interventional devices for priming to reduce the pressure needed to prime the catheter and reduce internal interference. The catheter being primed may be separated from the interventional devices within the lumen of the catheter by proximally retracting the interventional devices within the lumen of the catheter. For example, the interventional devices within the lumen of the catheter being primed can be proximally retracted from the catheter being primed as far as possible while still maintaining a nested or stacked relationship (e.g., at least about 2 cm or 5 cm or more axial overlap) in order to minimize the pressure needed to prime the catheter and minimize internal interference. In other words, a catheter can be separated from more proximal interventional devices for priming while a distal tip of an adjacent proximal interventional device is still positioned within the lumen of the catheter. Maintaining at least some of the distal tip of an adjacent proximal interventional device within the lumen of the catheter may allow for easier reinsertion and advancement of the proximal interventional device after priming.

In some embodiments, the axial overlap may be between about 2 cm and about 20 cm, between about 2 cm and 10 cm, between about 2 cm and 5 cm, between about 5 cm and 20 cm, between about 5 cm and 10 cm, or any other suitable range. In some embodiments, the axial overlap may be at least about 2 cm, at least about 5 cm, at least about 10 cm, at least about 20 cm, no more than 2 cm, no more than 5 cm, no more than 10 cm, no more than 20 cm, about 2 cm, about 5 cm, about 10 cm, about 20 cm, or any other suitable amount. For example, in some embodiments the axial overlap may be about 2 cm, 3 cm, 4 cm, 5 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, plus or minus ½ cm.

In some embodiments, the robotic drive table can be programed to proximally retract the inner interventional device(s) from the catheter being primed as much as possible while still maintaining a nested or stacked relationship. In other embodiments, the robotic drive table can be programmed to separate inner devices from the catheter being primed to a distance sufficient to optimize the length of the unobstructed lumen and result in an amount of fluid resistance lower than a threshold value. After the catheter being primed is separated from the other interventional devices, the catheter can be primed by flushing the catheter with fluid, such as saline, contrast media, or a mixture of saline and contrast media.

After the catheter is primed, it may be returned to an initial position and a next catheter of the interventional device assembly can be separated from the other interventional devices within its lumen for priming. This sequence can be repeated for each catheter of the interventional device assembly. In other embodiments, after a catheter is primed, it may be advanced to a ready or drive position to begin insertion into the patient. While the foregoing describes separating catheters to be primed by retraction of inner interventional devices, an outer catheter may also be separated from inner interventional devices by distally axially advancing the outer catheter relative to the inner interventional devices.

While fluid is being introduced under pressure into the proximal end of the annular lumen (e.g., into a hub of the outer catheter or a hemostasis valve coupled thereto), the inner catheter may be moved with respect to the outer catheter, to disrupt the holding forces between the microbubbles and adjacent wall and allow the bubbles to be carried downstream and out through the distal opening of the lumen or removed via aspiration. The catheters may be moved axially, rotationally or both with respect to each other. In certain embodiments, the catheters may be reciprocated axially, rotationally, or both with respect to each other. In some embodiments, the catheters may be moved intermittently axially, rotationally, or both. In other embodiments, the catheters may be rotated continuously or in a constant direction. In some embodiments, the catheters are moved using a driving mechanism that moves the catheter hubs, for example, a magnetically coupled drive system

In some implementations, a first catheter is moved reciprocally with respect to an adjacent catheter or guidewire such as axially over a stroke length in a range of from about 1 mm to about 250 mm, from about 10 mm to about 250 mm, from about 5 mm to about 125 mm, from about 25 mm to about 125 mm, from about 10 mm to about 50 mm, from about 15 mm to about 30 mm, from about 5 mm to about 30 mm, from about 15 mm to about 25 mm, from about 20 mm to about 40 mm, or any other suitable range. In some implementations, a first catheter is moved reciprocally with respect to an adjacent catheter or guidewire such as axially over a stroke length of at least 5 mm, at least 10 mm, at least 15 mm, at least 20 mm, at least 25 mm, at least 30 mm, at least 50 mm, no more than 10 mm, no more than 20 mm, no more than 25 mm, no more than 30 mm, no more than 50 mm, no more than 125 mm, no more than 150 mm, about 5 mm, about 10 mm, about 15 mm, about 20 mm, about 25 mm, about 30 mm, about 50 mm, or any other suitable stroke length.

In some implementations, a first catheter is moved reciprocally with respect to an adjacent catheter or guidewire such as axially at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as axially at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In one implementation, a first catheter is moved reciprocally with respect to the adjacent catheter or guidewire such as axially over a stroke length in a range of from about 0.5 inches to about 10 inches, or from about one inch to about 5 inches at a reciprocation frequency of no more than about 5 cycles per second or two cycles per second or less.

In some implementations, a first catheter is moved reciprocally with respect to an adjacent catheter or guidewire such as rotationally over an angle of rotation per stroke in a range of from about 5 degrees to about 180 degrees, from about 5 degrees to about 360 degrees, from about 15 degrees to about 180 degrees, from about 15 degrees to about 150 degrees, from about 15 degrees to about 120 degrees, from about 15 degrees to about 90 degrees, form about 15 degrees to about 60 degrees, from about 15 degrees to about 30 degrees, from about 30 degrees to about 180 degrees, from about 30 degrees to about 150 degrees, from about 30 degrees to about 120 degrees, from about 30 degrees to about 90 degrees, form about 30 degrees to about 60 degrees, from about 60 degrees to about 180 degrees, from about 60 degrees to about 150 degrees, from about 60 degrees to about 120 degrees, from about 60 degrees to about 90 degrees, from about 90 degrees to about 180 degrees, from about 90 degrees to about 150 degrees, from about 90 degrees to about 120 degrees, from about 120 degrees to about 180 degrees, from about 120 degrees to about 150 degrees, from about 150 degrees to about 180 degrees or any other suitable range. In some implementations, a first catheter is moved reciprocally with respect to an adjacent catheter or guidewire such as rotationally over an angle of rotation per stroke of at least 5 degrees, at least 15 degrees, at least 30 degrees, at least 60 degrees, at least 90 degrees, at least 120 degrees, at least 150 degrees, at least 180 degrees, at least 360 degrees, no more than 5 degrees, no more than 15 degrees, no more than 30 degrees, no more than 60 degrees, no more than 90 degrees, no more than 120 degrees, no more than 150 degrees, no more than 180 degrees, no more than 360 degrees, about 5 degrees, about 15 degrees, about 30 degrees, about 60 degrees, about 90 degrees, about 120 degrees, about 150 degrees, about 180 degrees, about 360 degrees, or any other suitable angle.

In some implementations, a first catheter is moved reciprocally with respect to an adjacent catheter or guidewire such as rotationally at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as rotationally at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In some implementations, a first catheter is moved reciprocally with respect to an adjacent catheter or guidewire for a number of reciprocations between 1 and 200, between 1 and 100, between 1 and 50, between 1 and 25, between 1 and 15, between 1 and 10, between 1 and 5, between 5 and 25, between 5 and 15, between 5 and 10, or any other suitable range. In some implementations, a first catheter is moved reciprocally with respect to an adjacent catheter or guidewire for at least 1 reciprocation, at least 2 reciprocations, at least 5 reciprocations, at least 10 reciprocations, at least 15 reciprocations, at least 25 reciprocations, at least 50 reciprocations, no more than 5 reciprocations, no more than 10 reciprocations, no more than 15 reciprocations, no more than 25 reciprocations, no more 50 than reciprocations, no more than 100 reciprocations, no more than 200 reciprocations, about 1 reciprocation, about 2 reciprocations, about 5 reciprocations, about 10 reciprocations, about 25 reciprocations, about 50 reciprocations, about 100 reciprocations, about 200 reciprocations, or any other suitable number. One reciprocation can include a movement (axially or rotationally) from a first position to a second position followed by a return from the second position to the first position.

In some implementations, a first catheter is moved reciprocally or rotationally with respect to an adjacent catheter or guidewire over a length of time in a range of from 1 about second to about 60 seconds, from about 1 second to about 45 seconds, from about 1 second to about 30 seconds, from about 1 second to about 20 seconds, from about 1 second to about 15 seconds, from about 1 second to about 10 seconds, from about 5 seconds to about 45 seconds, from about 5 seconds to about 30 seconds, from about 5 seconds to about 20 seconds, from about 5 seconds to about 15 seconds, from about 5 seconds to about 10 seconds, from about 10 seconds to about 30 seconds, form about 10 seconds to about 20 seconds, or any other suitable range. In some implementations, a first catheter is moved reciprocally with respect to an adjacent catheter or guidewire over a length of time of at least 1 second, at least 5 seconds, at least 10 seconds, at least 15 seconds, at least 20 seconds, at least 30 seconds, at least 45 seconds, at least 60 seconds, no more than 5 seconds, no more than 10 seconds, no more than 15 seconds, no more than 20 seconds, no more than 30 seconds, no more than 45 seconds, no more than 60 seconds, about 5 seconds, about 10 seconds, about 15 seconds, about 20 seconds, about 30 seconds, about 45 seconds, about 60 seconds, or any other suitable length of time.

Reciprocation of adjacent catheters to disrupt microbubbles may be accomplished manually by grasping the corresponding catheter hubs and manually moving the catheters axially or rotationally with respect to each other while delivering pressurized fluid (e.g., saline, contrast media, or a mixture of saline and contrast media). Alternatively, such as in a robotically driven system, a processor may be configured to robotically drive at least one of two adjacent catheter hubs (for example, at least one of hub 2914 and hub 2912) to achieve relative movement between the adjacent catheters thereby disrupting and expelling microbubbles, such as in response to user activation of a flush control. For example, in certain embodiments, two adjacent interventional devices may be moved relative to one another in response to a control signal from a control system. In certain embodiments, delivery of pressurized fluid may be performed in response to a control signal from a control system.

The reciprocation of adjacent catheters may generate shear forces that dislodge the air bubbles. For example, relative movement of the inner and outer surfaces of adjacent catheters may increase the fluid shear rate between the adjacent catheters during priming in comparison to static surfaces. In some embodiments, the shear force can be increased by increasing the flow rate of the solution (e.g., saline, contrast media, or a mixture of saline and contrast media) being provided by the fluidics system. In certain embodiments, both flow rate and relative movement between adjacent catheters are controlled to dislodge air bubbles.

In some embodiments, after each catheter is primed by the fluidics system, an ultrasound bubble detector may be used to confirm that the catheters are substantially free of air bubbles. For example, an ultrasound chip (such as mounted within a hub adjacent a catheter receiving lumen) may be run along the length of the catheters to confirm that no air bubbles remain in the system.

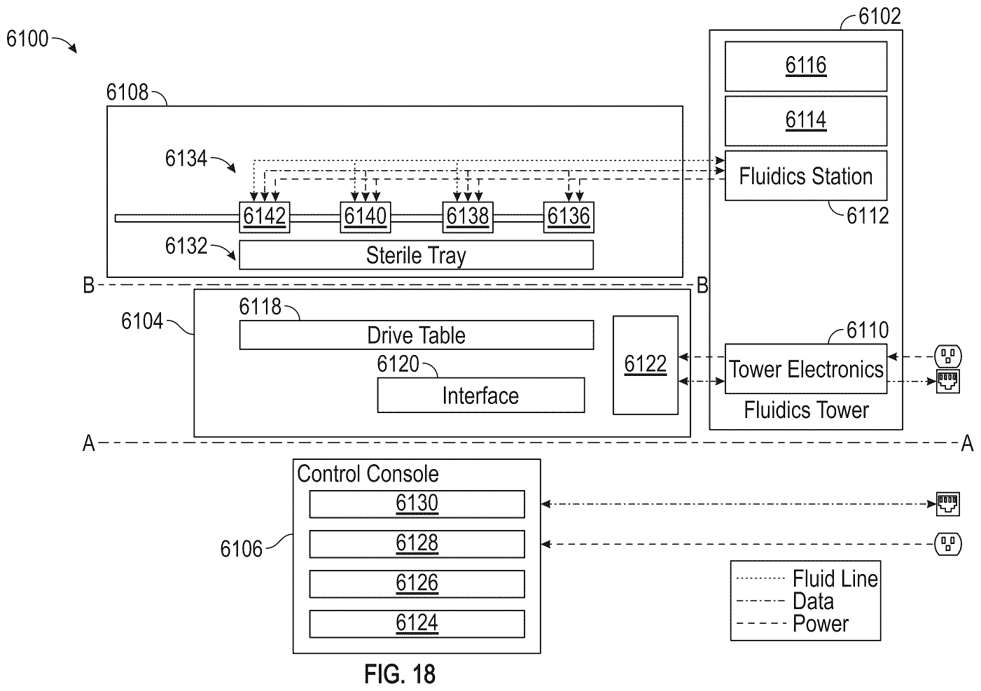

FIG. 18 illustrates a system diagram of an example of a medical device operation system 6100. The system diagram shows fluid and electrical connectivity between the subsystems of the medical device operation system 6100. The medical device operation system 6100 may include a fluidics tower 6102, a robotic drive system 6104, a control system 6106, and one or more interventional devices 6108.

The fluidics tower 6102 may be a housing or console including a fluidics management system for controlling the administration or removal of contrast, saline and/or bodily fluids to and/or from an interventional device. The fluidics tower 6102 may further include an electronics tower 6110, a fluidics station (or “system”) 6112, a monitor 6114, and one or more communication devices 6116. Although illustrated in FIG. 18 as part of the fluidics tower 6102, in other embodiments the electronics tower 6110 may be housed separately from the fluidics tower 6102 while still being in communication with the fluidics tower 6102, the interventional devices 6108, the robotic drive system 6104, and the control console system.

The electronics tower 6110 may be a housing configured to contain system electronics such as one or more processors and memory. The one or more processors and memory may be organized into one or more computer devices. The electronics tower 6110 may include a power cord configured to be operatively coupled to a power source, such as a battery, a generator, or an outlet. In some embodiments, the electronics tower 6110 may draw power from a source providing 110/220 volts of alternating current (VAC) power.

The system electronics may be a central hub for the medical device operation system 6100 interconnecting the electronic devices from other components as described in greater detail below. The system electronics of the electronics tower 6110 may be configured to transmit and receive electronic signals and/or data to operate components of the medical device operation system 6100. The electronics tower 6110 may include means for connecting to other devices. The electronics tower 6110 may transmit and/or receive electronic signals and/or data wirelessly or over a wired connection. In some embodiments, the electronics tower 6110 may include an ethernet port for connecting the system electronics to a network. In some embodiments, the electronics tower 6110 may include one or more ports for tethering to nearby electronic devices via a wired connection. For example, the electronics tower 6110 may have ports to run cables between the fluidics tower 6102 and the robotic drive system 6104 and/or control system 6106. Alternatively, the electronics tower 6110 may be configured to connect wirelessly to nearby electronic devices. For example, the electronics tower 6110 may include a personal area network (PAN) module, such as Bluetooth®, or other network capabilities to transmit data wirelessly.

In some embodiments, electronic signals and/or data may include instructions for a system, subsystem, component, or device to perform a particular task. Additionally and/or alternatively, electronic signals and/or data may include indicators or data measured from sensors for processing by a computing device.

In some embodiments, the electronics tower 6110 may connect to other devices over a communication network (“network”). The network may cover a small geographic area such as a particular room or building, a medium geographic area such as a city, or a large geographic area so long as there is access to a network. For example, the network may be a local area network (LAN) or wireless local area network (WLAN) including a series of devices linked together to form a network within a hospital or clinic. Alternatively, the network may be a wide-area network (WAN) including a series of devices linked together to form a network within a medical campus including two or more buildings. Alternatively, the network may be an intranet or internet for providing global connectivity. Connecting over the network advantageously connects the operating room to physicians located around the world, including experts located across the nation or in other countries, without requiring the physician to travel to the operating room. This advantageously connects patients to physicians without the time or cost required for the physician to physically travel to the operating room. In some procedures, every minute of delay before a procedure is performed can increase the chance of a bad outcome, and thus such surgical systems can help mitigate damage to the patient due to a delay in starting the surgical procedure.

The fluidics system 6112 may include one or more subsystems including one or more containers, one or more tubes, and one or more pumps. The subsystems may be divided and organized into a contrast subsystem, a saline subsystem, and/or an aspiration (or “vacuum”) subsystem. The fluidics system 6112 may be the fluidics system described herein.

A contrast subsystem may be configured for supplying contrast to a patient. The contrast subsystem may include one or more containers for storing and supplying contrast, one or more fluid communication channels (“tubes”), one or more valves, and a high-pressure pump.

A saline subsystem may be configured for supplying saline to a patient. The saline subsystem may similarly include one or more containers for storing and supplying saline, one or more tubes, one or more valves, and one or more pumps.

An aspiration subsystem may be configured for removing biological material from a patient. The aspiration subsystem may include one or more containers, one or more tubes, one or more valves, and a vacuum pump.

The one or more pumps and containers of the contrast, saline, and aspiration subsystems may be contained with the fluidics tower 6102. The one or more tubes of the contrast, saline, and aspiration subsystems may extend out of the fluidics tower 6102 for interacting with and coupling to other devices.

The monitor 6114 may be any electronic visual computer display (or displays) that includes a screen and circuitry configured to interpret electronic signals to display one or more images. For example, the monitor 6114 may include an imaging window, a speed indicator, a rotational indicator, an axial position bar, a telescopic position window, one or more axial position indicators, and/or other graphical user interfaces or windows. In some embodiments, the monitor 6114 may be configured to display fluoroscopic images, catheter data, fluidics information (e.g., information relating to a contrast injection subsystem including its current operation status, information relating to a saline subsystem including its current operation status, and/or information relating to a aspiration subsystem including its current operation status) including current state information) providing saline, providing vacuum for aspiration), and patient data including vital signs. In some embodiments, the monitor 6114 may be the display 23 described above.

The one or more communication devices 6116 may be one or more microphones, one or more cameras, and/or one or more audio output devices such as a speaker and/or a headset.

The fluidics system 6112, the monitor 6114, and the one or more communication devices 6116 may be electrical communication with the electronics tower 6110 and configured to receive and/or transmit electronic signals and/or data therebetween. In some embodiments, the electronic signals and/or data may include instructions to activate one or more pumps and/or one or more valves of the fluidics system 6112. For example, the instructions may direct the fluidics system 6112 to provide saline and/or contrast to the one or more interventional devices 6108. In some embodiments, the data may include video and/or audio inputs and audio outputs for the monitor 6114 and one or more communication devices 6116. For example, the data may be one or more images to be displayed on the monitor 6114 and/or audio-visual data captured by the one or more communication devices 6116.

The fluidics tower 6102 may be further configured to be operatively coupled with the one or more interventional devices 6108. In some embodiments, the fluidics system 6112 may be mechanically coupled to and/or in fluid communication with the one or more interventional hubs 6134. Accordingly, activating the fluidics system 6112 may provide contrast, saline, and/or suction to the interventional hubs 6134 and corresponding interventional devices.

The robotic drive system 6104 may include a plurality of components to drive one or more access systems such as catheters and guidewires during a procedure. The robotic drive system 6104 may be the drive system 18 described above. The robotic drive system 6104 may include a drive table 6118, an interface 6120, and a joint setup 6122.

The drive table 6118 may support the one or more disposable devices 6108 (e.g., a catheter) configured to be advanced to access a patient for performing a surgical procedure and/or for introducing saline, contrast media or therapeutic agents, or providing aspiration. The drive table 6118 may further support a sterile barrier.

The drive table 6118 may be the support table 20 described above. The drive table 6118 may be positioned over or alongside a patient, and configured to axially advance, retract, and in some cases rotate two or three or more different concentrically oriented intravascular devices of an interventional device assembly. The drive table 6118 may include electronics and motors for controlling the location of the interventional devices and actuation of fluidics components.

In some embodiments, the drive table 6118 may include one or more hub adapters. The one or more hub adapters may include the drive magnets described above. Movement of the drive magnets may be driven by a drive system carried by the drive table 6118. Movement of the drive magnets may be configured to drive one or more interventional hubs 6134 of the one or more disposable devices 6108. The drive table 6118 and the one or more disposable devices 6108 may be separated such that the one or more interventional hubs 6134 may not mechanically couple to the drive table 6118 as shown by axis B-B.

The interface 6120 may be any device configured to interact with and/or display information to personnel locally situated within an operating room during a procedure, such as a bedside user. For example, a bedside user may be nurse or surgical technician staffed within the operating room. The interface 6120 may include an imaging window, a speed indicator, a rotational indicator, an axial position bar, a telescopic position window, and/or one or more axial position indicators. The interface 6120 may be the display 23 described above configured to display fluoroscopic images, catheter data, pressure values of the fluidics system, and/or other patient data. In some embodiments, the interface 6120 may be a touchscreen device such as a tablet computer. The interface 6120 may display information to a bedside user. The information displayed to the bedside user may include directions and/or prompts for the bedside user to follow. For example, the information may describe what steps to perform next, how to position the robotic drive system 6104, when to deploy the drapes, whether the system is malfunctioning or whether an error is detected, and/or prompt the bedside user to otherwise interact with the system. In some embodiments, the interface 6120 is configured to accept user input to control one or more components, for example, position of the drive table.

The interface 6120 may be in communication with one or more portions of the medical device operation system 6100 (for example, the robotic drive system 6104, the fluidics tower 6102, the disposable devices 6108, etc.). In some embodiments, the interface 6120 may be mechanically coupled to the robotic drive system 6104, be housed separately, or be mechanically coupled to another part of the medical device operation system 6100. The interface 6120 may control the joint setup 6122 of the robotic drive system 6104. For example, the interface 6120 may control the transition processes between a storage position and a deployed position, engaging a priming sequence, or controlling fine motor adjustments for providing minor adjustments to the positions of the interventional hubs. Controlling the joint setup 6122 and motors with the interface 6120 advantageously provides greater precision and setup before an operation by individuals present in the operating room.

The interface 6120 may advantageously provide a backup control mechanism to interact with and provide input to control the medical device operation system 6100, for example, in the event that the control system 6106 is rendered incapable of performing an operation.

The joint setup 6122 may include a plurality of joints and motors for controlling the positioning and movements of the robotic drive system 6104. In some embodiments, the joint setup 6122 may initialize the robotic drive system 6104 into a starting position. The initialization process may include transitioning the robotic drive system 6104 from a storage position to a deployed position and vice versa. For example, the joint setup 6122 may be configured to transition the robotic drive system 6104 from a storage position the robotic drive system 6104 to a deployed position such that at least a portion of the robotic drive system 6104 transitions from a compact state to a position where at least a portion of the robotic drive system 6104 is positioned either over or alongside a patient.

Within the robotic drive system 6104, the drive table 6118 may be mechanically coupled with the interface 6120 and the joint setup 6122. The joint setup 6122 may also be electrically connected to the drive table 6118 and the interface 6120. The robotic drive system 6104 may be configured for the joint setup 6122 to transmit electronic signals and data to the drive table 6118 and the interface 6120. Additionally and/or alternatively, the robotic drive system 6104 may be configured for the joint setup 6122 to receive electronic signals and data from the drive table 6118 and the interface 6120.

The control system 6106 may be a collection of components configured to control and operate the robotic control system described above. In some embodiments, the control system is a control console or is coupled to a control console. The control system 6106 may further include an operator controller 6124, an interface 6126, a monitor 6128, and one or more communication devices 6130. The control system 6106 may be locally positioned or remotely positioned. For example, in some embodiments, the control system 6106 may be located in the operating room with the fluidics tower 6102, the robotic drive system 6104, and the one or more disposable devices 6108. Alternatively, the control system 6106 may be located remotely (e.g., in a control room) as illustrated by line A-A. The control system 6106 may include system electronics including one or more processors and one or more memory components (“memory”). The system electronics may be configured to electrically connect the controller 6124, the interface 6126, the monitor 6128, and the one or more communication devices 6130.

The control system 6106 may include means for connecting to other devices. The control system 6106 may transmit and/or receive electronic signals and/or data wirelessly or over a wired connection. In some embodiments, the control system 6106 may include an ethernet port for connecting the system electronics to a network. In some embodiments, the control system 6106 may include one or more ports for tethering to nearby electronic devices via a wired connection. For example, the control system 6106 may have ports to run cables between the control system 6106 and the fluidics tower 6102 and/or robotic drive system 6104. Alternatively, the control system 6106 may be configured to connect wirelessly to nearby electronic devices. For example, the control system 6106 may include a Bluetooth® module or other network capabilities to transmit data wirelessly.

In some embodiments, the control system 6106 may connect to other devices over a network as described above.

The controller 6124 may be any device configured to enable a surgeon to control portions of the medical device operation system 6100 in the same location as the patient. For example, the controller 6124 may be any of the control mechanisms or controllers described herein. The controller 6124 may enable a user to control portions of the fluidics tower 6102, the interventional devices 6108, and the robotic drive system 6104. For example, the controller 6124 may be configured to move the to desired positions to perform a procedure on a patient as described herein.

The controller 6124 may be part of the control system 6106 or connected, wirelessly or via a wired connection, to the control system 6106.

The interface 6126 may be configured to display information to the surgeon. The interface 6126 may be the display 23 described above configured to display fluoroscopic images, catheter data, or other patient data. The interface 6126 may be a touchscreen device. The interface 6126 be a graphical user interface.

The monitor 6128 may include one or more electronic displays. The monitor 6128 may be any electronic visual computer display that includes a screen and circuitry configured to interpret electronic signals to display one or more images. The monitor may display the interface 6126. In some embodiments, the monitor 6128 may be configured to display fluoroscopic images, catheter data, or other patient data. Alternatively, the monitor 6128 may be configured to display one or more views of the operating room. For example, the monitor 6128 may be configured to display the working area during a procedure by displaying only the surgical site. In another example, the monitor 6128 may display the entire operating room including the surgical technicians. In another example, the monitor 6128 may display more than one view. Displaying a plurality of views to capture the entire operating room may advantageously enhance communication and understanding between the physician and the technicians and/or assistants located in the operating room thereby increasing the efficiency and safety of procedures.

The one or more communication devices 6130 may be any one or more microphones, one or more cameras, and/or one or more audio output devices.

As shown in FIG. 18, the fluidics tower 6102, the 6104, the control system 6106, and the one or more disposable devices 6108 are connected to a power source. In some embodiments, the fluidics tower 6102 and the control system 6106 may be directly connected to a power source such as an outlet. In some embodiments, the robotic drive system and the one or more disposable devices 6108 may indirectly connect to a power source. For example, the robotic drive system 6104 and the one or more disposable devices 6108 may receive power from the fluidics tower 6102. In such embodiments, the joint setup 6122 of the robotic drive system 6104 may be electrically connected to the electronics tower 6110 of the fluidics tower and the interventional hubs 6134 of the one or more disposable devices 6108 may be electrically connected to the fluidics system 6112 of the fluidics tower 6102 such that power may be transmitted therebetween.

Furthermore, as shown in FIG. 18, the fluidics tower 6102, the robotic drive system 6104, the control system 6106, and the one or more disposable devices 6108 may be electrically connected and configured to share electrical signals and/or data. In some embodiments, the fluidics tower 6102 may be electrically connected and configured to share electrical signals and/or data with the robotic drive system 6104, the control system 6106, and the one or more disposable devices 6108. For example, the electronics tower 6110 of the fluidics tower 6102 may be electrically connected with the joint setup 6122 of the robotic drive system 6104 and the control system 6106 while the fluidics system 6112 of the fluidics tower 6102 may be electrically connected with the one or more interventional hubs 6134 of the one or more disposable devices 6108. In some embodiments, the electronics tower 6110 may be electrically connected with the control system 6106 via a network, as shown in FIG. 18.

The one or more interventional hubs 6134 may include a first interventional hub 6136, a second interventional hub 6138, a third interventional hub 6140, and a fourth interventional hub 6142. In some embodiments, the one or more interventional hubs 6134 may be aligned sequentially such that the first interventional hub 6136 may be positioned at a first end and the fourth interventional hub 6142 may be positioned at a second end opposite the first end. In some embodiments the first end may be a proximal end closest to a patient and the second end may be a distal end furthest from the patient. A sterile tray 6132 may separate the one or more interventional hubs 6134 and corresponding interventional devices from a support table. In some embodiments, the sterile tray 6132 forms a sterile barrier, such as the sterile barrier 32 described above.

In some embodiments, the first interventional hub 6136 may be a guidewire hub, such as the guidewire hub 26 described above; the second interventional hub 6138 may be a first catheter hub, such as the access catheter hub 2910 described above; the third interventional hub 6140 may be a second catheter hub configured to engage with and guide a procedure catheter, such as the procedure catheter hub 2912 described above; and the fourth interventional hub 6142 may be a third catheter hub configured to engage with and guide a guide catheter, such as the guide catheter hub 2914. In some embodiments, the guide catheter may extend distally from the fourth interventional hub 6142.

The fluidics tower 6102 may be electrically connected to the robotic drive system 6104, the control system 6106, and the one or more disposable devices 6108 wherein electrical signals and/or data may be transmitted between therebetween as discussed in greater detail below. The local system may transmit information about the fluidics system 6112, the robotic drive system 6104, and the plurality of interventional devices 6108 to the control system 6106 via the fluidics tower 6102.

The one or more communication devices 6130 may be in electrical communication with a power source. The one or more communication devices 6130 may further be in electrical communication with the electronics tower 6110 and configured to receive and/or transmit electronic signals and/or data therebetween. In some embodiments, the one or more communication devices 6130 is in electrical communication with the electronics tower 6110 via a network. For example, the one or more communication devices 6130 may be electrically coupled to an ethernet cable configured to connect the one or more communication devices 6130 to a network, wherein the electronics tower 6110 may be electrically coupled to the network.

### Hub Assembly:

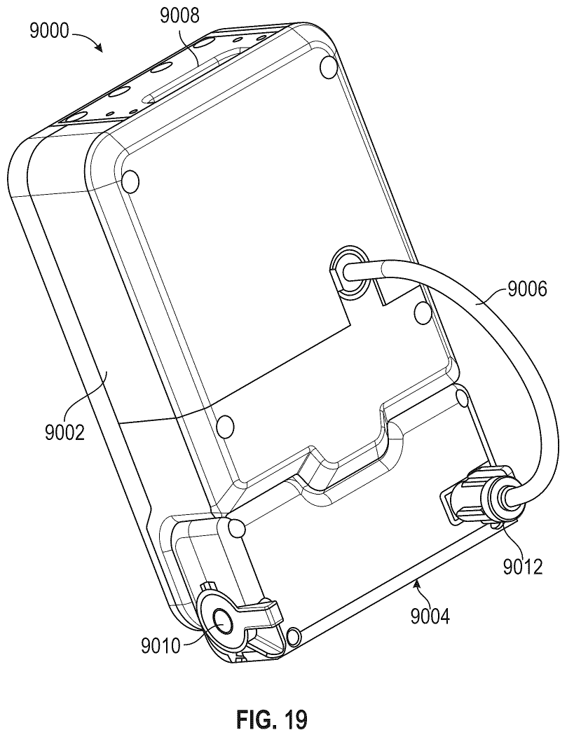

FIG. 19 illustrates a hub assembly 9000. The hub assembly 9000 may include any of the same or similar features and/or functions as the other embodiments of hubs and hub assemblies described herein and vice versa.

In some embodiments, the hub assembly 9000 can include a first subassembly, puck, or mount 9002 and a second subassembly or hub 9004. The mount 9002 can also be referred to as a catheter puck, a hub mount, and/or a first hub member. The mount 9002 can be configured to couple to and move along a drive table. The hub assembly 9000 can be configured to be positioned on a sterile side (e.g., a disposable equipment side) of a sterile barrier.

In some embodiments, the hub 9004 can be referred to as a second hub member. The hub 9004 may include or couple to an interventional device, such as a catheter or guidewire.

As described herein, in certain embodiments an interventional device may be coupled to a fluidics management system (e.g., to receive fluids such as contrast or saline, or for aspiration). In some embodiments, the mount 9002 can be coupled to the fluidics management system. In some embodiments, a fluidics connector 9006 can extend between and fluidly couple the mount 9002 and the hub 9004.

The mount 9002 can further include a first housing. The first housing can define one or more openings 9008 and a plurality of internal components described in greater detail below. The first housing can form an outer shell to protect the internal components of the mount 9002. The first housing can include at least one side shaped and/or dimensioned (e.g., having a contour) for receiving the hub 9004.

The one or more openings 9008 can provide access for fluidics and/or electrical connections into the mount 9002. In some embodiments, a contrast tube, a saline tube, and/or an aspiration tube may extend through the one or more openings 9008 into the mount 9002. Additionally, in some embodiments, a power line may extend through the one or more openings 9008 to provide electrical power into the mount 9002. The mount 9002 can be configured to receive an input from one or more active torque elements of an active torque subsystem. In some embodiments, the inputs from the one of more active torque elements may be a magnetic rotary force as described herein. The mount 9002 can be configured to transmit one or more outputs to the hub 9004. In some embodiments, the mount 9002 may transform one or more rotary inputs of the one or more active torque elements into corresponding linear and/or rotary outputs. In some embodiments, the mount 9002 may be configured to translate linearly along a drive table (e.g., in response to linear movement of hub adapter within the drive table due to a magnetic coupling between mount 9002 and the hub adapter).

The hub 9004 can further include a second housing. The hub 9004 can include a lumen 9010 for receiving an interventional device therein. The hub 9004 can include a luer 9012. The second housing can form an outer shell to protect the internal components of the hub 9004. In some embodiments, the second housing may include at least one side shaped and/or dimensioned (e.g., having a contour) to correspond to shape of the first housing. For example, the contour of the second housing can correspond to the contour of the first housing of the mount 9002. The hub 9004 can be configured to receive one or more inputs from the mount 9002. The hub 9004 can be configured to transmit one or more outputs. In some embodiments, the hub 9004 may transform the outputs of the mount 9002 into corresponding linear and/or rotary motion of components within or coupled to the hub 9004 (e.g., the interventional device coupled to the hub 9004 and/or one or more fluidics components).

The fluidics connector 9006 can be a tubular body defining an interior lumen extending from one end of the fluidics connector 9006 to a second end of the fluidics connector 9006. In some embodiments, the fluidics connector 9006 may be configured to transport fluids between the mount 9002 and the hub 9004. For example, the fluidics connector 9006 may facilitate the flow of contrast, saline, bodily fluids, and/or air between the mount 9002 and the hub 9004. The fluidics connector 9006 can transport fluids from the mount 9002 to the hub 9004, or vice versa. The fluidics connector 9006 may form an airtight seal.

The hub 9004 may be removably coupled to the mount 9002. In some embodiments, the hub 9004 can be mounted to a mounting element defined by the mount 9002. The fluidics connector 9006 may be coupled to both the mount 9002 and the hub 9004. In some embodiments, the hub 9004 may be in fluid communication with the mount 9002 via the fluidics connector 9006. Accordingly, fluids may be transferred between the mount 9002 and the hub 9004 via the fluidics connector 9006.

In certain embodiments, a mount 9002 can include at least a portion of a passive torque subsystem. In certain embodiments, a portion of a passive torque subsystem may be included in a mount 9002, and another portion of a passive torque subsystem may be included in a hub 9004 coupled to the mount 9002.

Additional details regarding a hub assemblies can be found in U.S. patent application Ser. No. 18/986,519, entitled ROBOTIC HUB ASSEMBLY, filed Dec. 18, 2024, which is hereby expressly incorporated by reference in its entirety herein. Drive Assembly:

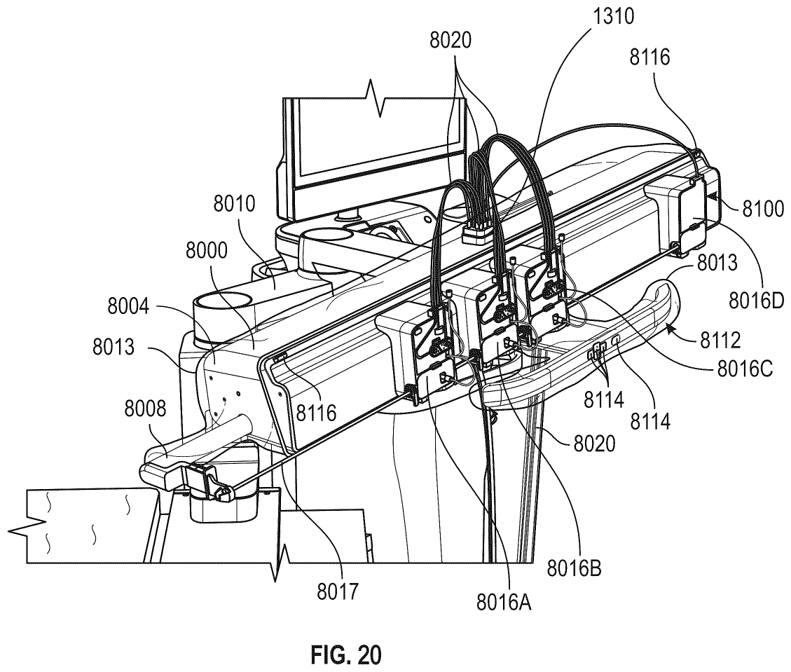

FIG. 20 illustrate an embodiment of a robotic drive system having a drive table 8000.

The drive table 8000 may operate in the same or similar manner as described herein for other embodiments.

The drive table 8000 can include a main body 8004. In some embodiments, the drive table 8000 can include an extendable or telescoping member 8008. The telescoping member may be a telescoping arm or a telescoping support.

As shown in FIG. 20, the main body 8004 can be a longitudinal body having one or more walls defining an interior cavity. One of the one or more walls of the main body 8004 can be a planar support surface. The interior cavity can be configured to house and/or support the telescoping member 8008. The interior cavity can further include a drive system. The drive system can include one or more hub adapters, which may have magnets for magnetically driving one or more hub assemblies.

The telescoping member 8008 can be a longitudinal body configured to extend from and/or retract into the main body 8004. In some embodiments, the telescoping member 8008 can be a support bracket. In some embodiments, the telescoping member 8008 may be configured to support one or more interventional devices at a position proximal to a patient. For example, as shown in FIG. 20, an interventional device 8017 can extend from the one or more mounts or hub assemblies through a distal end of the telescoping member 8008. In some embodiments, the telescoping member 8008 (e.g., distal end) can be coupled to an access sheath (e.g., a femoral access sheath) at a patient access point. In some embodiments, the robotic drive system can also include an anti-buckling feature (which may be the same or similar to any of the anti-buckling features described herein) that can couple to the distal end of the telescoping member 8008. For example, the anti-buckling feature may extend from a hub assembly to the distal end of the telescoping member 8008. The anti-buckling feature can be configured to stiffen a portion of an interventional device supported by the drive table 8000.

The telescoping member 8008 may be used to reduce the size of the drive table 8000. In embodiments without a telescoping member, a drive table may move along a linear rail that extends along an entire desired range of travel for the drive table. For example, an eight foot linear rail may be used to provide a full range of motion for the drive table. Alternatively, a drive table may itself have an eight foot drive surface to accommodate a full range of motion for the interventional devices. Such linear rails and/or drive tables may be large and heavy. In contrast, a telescoping member 8008 may have a shorter length (e.g., four feet, three feet, two feet) than the above-described linear rail while allowing for the same range of motion of interventional devices positioned on the drive table 8000 (e.g., through movement of the telescoping member 8008 and/or movement of the main body 8004 relative to the telescoping member). Accordingly, the use of the telescoping member 8008 may reduce the size and mass of the drive table in comparison to alternative embodiments. In some examples, the drive table 8000 may be sufficiently long to provide an adequate travel distance for the interventional devices and may include a telescoping member 8008 to extend to a position adjacent to the access point of the patient. For example, the main body 8004 may be five feet in length and the telescoping member may be three feet in length.

The drive table 8000 can be further coupled to a base 8010. The base 8010 can be a device for supporting and/or orienting the drive table 8000 to a desired position. In some examples, the base 8010 can be a static structure to support the drive table 8000. In some embodiments, as shown in FIG. 20, the base 8010 can be a dynamic support system configured to selectively orient the drive table 8000 between two or more positions. For example, the base 8010 can include a robotic arm, such as a selective compliance articulated robot arm (SCARA). In some embodiments, the robotic arm can be configured to transition the drive table 8000 between a stowed configuration and one or more operational configurations. In some embodiments, the robotic arm can be configured to transition the drive table 8000 between a plurality of operational configurations. In some examples, the stowed configuration may correspond to a vertical orientation of the drive table 8000. In some examples, the one or more operational configurations may correspond to a horizontal or otherwise non-vertical orientation of the drive table 8000. The drive table 8000 can be configured to position one or more hub assemblies proximal to an access point on a patient.

The drive table can include a handle 8112. The handle 8112 can be configured to provide a grasping location to provide for manipulating the drive table 8000 (e.g., between stowed configuration and/or one or more operational configurations). In some embodiments, as shown in FIG. 20, the handle 8012 can extend from the main body 8004. In some embodiments, a force cell or load cell can be positioned within the handle or at a location where the handle couples to the main body 8004 or other portion of the drive table 8000. In some embodiments, forces applied by a user to the handle 8112 to move the drive table 8000 can be detected by the force cell or load cell, and can be processed by the control system. The control system can cause the robotic arm of the base 8010 to move in response to the detected forces, for example, to cause the drive table 8000 to move in accordance to the detected forces (e.g., in the same direction). In some examples, the orientation of the drive table 8000 may be aligned via the handle 8112.

In some embodiments, the handle 8112 may include a control surface. For example, the handle 8112 may include a display screen (such as display 23). In some embodiments, the handle 8112 may include a joystick or a directional pad having a plurality of buttons corresponding to a distinct direction. For example, the directional pad may include an up button, a down button, a right button, and a left button. The control surface may be used to drive the base 8010 of the drive table 8000 to orient and/or align the drive table 8000. Accordingly, a user may engage the control surface to actuate the base 8010. During alignment of the drive table 8000 and/or the base 8010, the drive table 8000 may never contact the patient. Avoiding the patient during alignment and set up may increase the safety of the use of the drive table 8000. The handle 8112 can include a plurality of buttons 8114. The buttons 8114 can be provided for manually operating the drive table 8000. In some examples, the buttons 8114 can include directional indicators. For example, the buttons 8114 can include a left directional indicator, a right directional indicator, an upward directional indicator, and a downward directional indicator. In some embodiments, the buttons 8114 can include additional indicators. The buttons 8114 may be used to select a desired hub assembly 8016A-D and to manually move the desired hub assembly 8016A-D along the drive surface. In some embodiments, the buttons 8114 used to move the drive table vertically, axially, and/or laterally. In some embodiments, the buttons 8114 may be used to navigate within a user interface (e.g., a user interface that is coupled to the handle or a user interface positioned remote from the handle within the operating room).

As shown in FIG. 20, one or more sterile coverings 8013 (e.g., sterile drapes or sleeves) can be applied to the drive table 8000. The one or more sterile coverings 8013 can be flexible sterile coverings. In some embodiments, a sterile covering 8013 can be applied to the main body 8004. In some embodiments, a sterile covering 8013 can be applied to the handle 8112. In some embodiments, a sterile covering 8013 can be applied to the base 8010. In some embodiments, a sterile covering 8013 can be applied to the telescoping member 8008. For example, the sterile covering 8013 can be connected to a distal portion of the telescoping member 8008 (and in some embodiments a portion of the main body 8004) so that the sterile covering 8013 extends with the telescoping member 8008 as the telescoping member 8008 extends from the main body 8004. In some embodiments, a sterile covering 8013 can be positioned between the drive table 8000 and the drive assembly 8014. In some embodiments, a sterile covering 8013 can be in the form of a tubular drape. In some embodiments, the drive assembly 8014 can additionally include sterile field barrier.

In some embodiments, a single sterile covering 8013 in the form of a tubular drape may be positioned over the main body 8004 and the telescoping member 8008 (e.g., coupled to a distal end of the telescoping member) so that the sterile cover 8013 moves to cover the length of the telescoping member 8008 as the telescoping member 8008 and main body 8004 move relative to one another. In some embodiments, the single sterile covering 8013 may also cover the handle 8112. In some embodiments, the single sterile covering 8013 may also cover the base 8010 (e.g., drive arms thereof). In other embodiments, separate sterile coverings 8013 may cover any of the telescoping member 8008, the main body 8004, handle 8012, and base 8010.

In certain embodiments, the drive system can include one or more hub assemblies 8016A-D. The hub assemblies 8016A-D and related interventional devices may operate in the same or similar manner as described above for other embodiments.

In certain embodiments, the drive system can include a sterile barrier 8100. In certain embodiments, the sterile barrier 8100 can removably couple to the drive table 8000. The sterile barrier 8100 can be contoured to fit on the drive table 8000 (e.g., the main body 8004). Accordingly, the profile of the sterile barrier 8100 can correspond to the profile of the drive table 8000. For example, the profile of the sterile barrier 8100 can correspond to the profile of the main body 8004. The sterile barrier 8100 may provide a drive surface for one or more hub assemblies 8016A-D.

The sterile barrier 8100 can further include one or more engagement surfaces to secure the sterile barrier 8100 to the drive table 8000 (e.g., to the main body 8004). For example, an engagement element such as an anchor may extend through the engagement surfaces.

The drive table 8000 can include one or more anchors 8116. The anchors 8116 may be positioned along a top surface of the main body 8004. The sterile barrier 8100 can be secured to the drive table 8000 via anchors 8116 of the drive table 8000 extending through the engagement surfaces. In an assembled state, the sterile barrier 8100 may be placed over a sterile covering or drape (e.g., sterile covering 8013). The sterile barrier 8100 may be rigid. The sterile covering or drape may be flexible. The hub assemblies 8016A-D may be positioned on the drive table 8000 (e.g., positioned on a drive surface of the sterile barrier 8100) after the sterile barrier 8100 is applied to the drive table 8000. Additionally, a splitter 1310 as described herein can be positioned on the drive table 8000. For example, as shown, the splitter 1310 can be positioned along a top surface of the main body 8004.

As described herein, or more of the hub assemblies 8016A-D can be coupled to a fluidics assembly. As shown in FIG. 20, one or more fluid conduits 8020 may couple one or more of the hub assemblies 8016A-D to the fluidics assembly or may couple components of the fluidics assembly to one another.

### Fluidics Assembly:

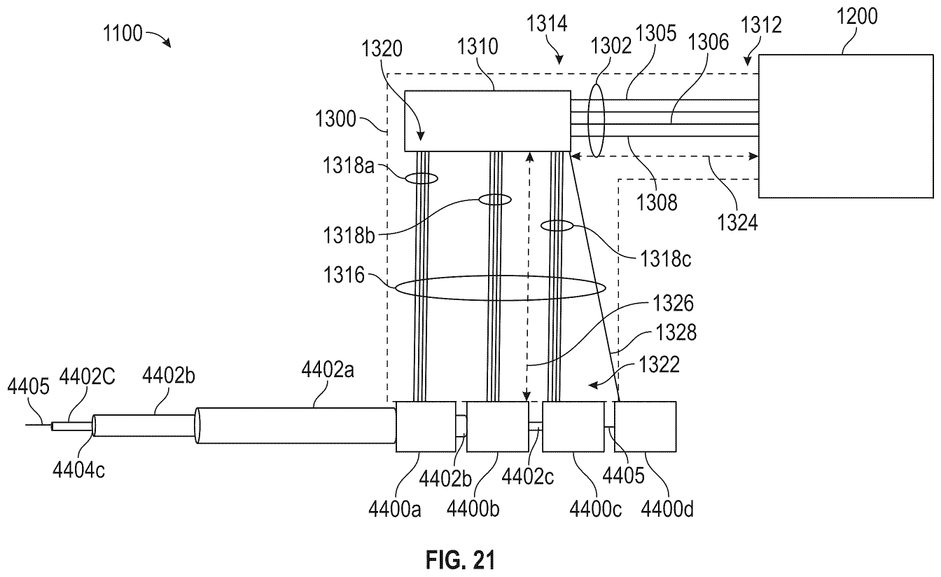

FIG. 21 is a schematic of the example of the fluidics assembly 1100. In FIG. 21, the fluidics assembly 1100 includes a cassette 1200 that couples to the pump station and to a saline source, a contrast source, and a vacuum source. The fluidics assembly 1100 also includes a plurality of fluid communication channels 1300 to provide saline, contrast, and vacuum to one or more mounts 4400a-c and to the hubs (not shown) and catheters 4402a-c coupled to the mounts 4400a-c. The fluid communication channels 1300 also can include a plurality of electrical leads that connect components in the mounts (e.g., bubble sensors, pressure sensors, etc.) to an electrical interface of the cassette to provide signals to the control system when the cassette 1200 is coupled to a pump station. In systems that include a plurality of hub assemblies, different hub assemblies may be configured differently or may have different fluids communicated to them, which are provided to a catheter. For example, in a system with a plurality of hub assemblies, one hub assembly may only provide saline to a catheter, and/or one hub assembly may provide saline and contrast to a catheter, and/or one hub assembly may provide saline and vacuum to a catheter, and/or one hub assembly may provide saline, contrast, and vacuum to a catheter.

One challenge of a fluidics system that provides fluids (e.g., saline and contrast) that enter a patient via a catheter is to provide such fluids in air-free (that is, bubble-free) fluid flows. Typically when the cassette, fluid communication channels, hubs/mounts, and catheters are provided for use in an operating room they are not filled with fluid, and thus require priming to remove air from the saline and contrast flow-paths to prevent air bubbles entering a patient during a medical procedure. Once primed, air bubble detectors in the cassette and the mounts can help mitigate risks associated with air bubbles within saline and contrast flow-paths. However, because of the dangerous consequences of air entering a patient's bloodstream, where it is possible the fluid communication channels are visually inspected for air bubbles by a medical partitioner after the saline and contrast flow-paths are primed. Because of flexibility and mobility requirements for providing fluids from static saline and contrast fluid sources across a certain distance to moving mounts/catheters, at least a portion of the fluid communication channels between the cassette and the mounts can include flexible clear tubing. During the visual inspection, each centimeter of all the clear tubing supplying saline or contrast from the cassette to the mounts 4400a-c is carefully inspected to determine if air bubbles are present, and if so the lines can be re-primed. The length of time it takes to inspect the clear tubing for air bubbles directly relates to the total length of the contrast and saline tubing between the cassette and the mounts. In some examples, the pump station is located several feet away from the set of mounts 4400a-c to provide room around the patient, and the saline and contrast fluid communication channels 1300 between the cassette 1200 and mounts 4400a-c may be about 14 feet long. If, as illustrated in FIG. 21, there are three mounts 4400a-c coupled to the communication channels 1300 and the communication channels include individual tubes for saline and contrast for each of three mounts 4400a-c, this results in 84 linear feet of saline and contrast tubing to inspect. To make inspection of the tubing easier, the communication channels can be configured in a “flat” ribbon-like design with the saline and contrast communication channels next to each other such that the contrast and saline tubing can be inspected at the same time, which effectively reduces the length of tubing to be inspected to 42 feet (14′ per mount). During the inspection, as the lines are each handled from end-to-end the medical practitioner typically will change gloves for each line (e.g., three glove changes). Such an inspection can take between 7 and 10 minutes, which is a long delay before proceeding with the operation when every minute of delay of performing a thrombectomy can result in further harm to the patient.

The first tubing set 1302 includes a one saline tube (channel) 1308 in fluid communication with a first saline flow-path in the cassette 1200 and a second saline flow-path in a splitter 1310, one contrast tube (channel) 1306 in fluid communication with a first contrast flow-path in the cassette 1200 and a contrast flow-path in the splitter 1310, and one vacuum tube (channel) 1305 in fluid communication with a first vacuum flow-path in the cassette 1200 and a second vacuum flow-path in the splitter 1310. The splitter 1310 provides saline, contrast, and vacuum from the first tubing set 1302 to the second tubing set 1316, and includes channels forming a second saline flow-path, a second contrast flow-path, and a second vacuum flow-path, where the second saline flow-path, the second contrast flow-path, and the second vacuum flow-path each having a plurality of branches structured to provide outputs of saline, contrast, and vacuum to each mount 4400a-c from the single inputs of saline, contrast, and vacuum from the first tubing set 1302. The first tubing set 1302 can be configured in a ribbon-like design. In some embodiments, the length 1324 of the first tubing set can be between about 5 feet and about 17 feet in length based on the specific embodiment. In some examples, the ratio of the first length to the second length is greater than about 1:2 (e.g., about 1:3, 1:4, 1:5, 1:6, of 1:7) to minimize the amount of tubing that needs to be visually inspected. In a specific embodiment, the length of the first tubing set is about 12 feet in length. The tubes in the first tubing set have inner diameters to suitably provide saline, contrast and vacuum to the catheters. As a nonlimiting examples, the inner diameter of saline and contrast tubes (channels) in the first tubing set and the second tubing set can be about 0.071 inches. As a nonlimiting example, the inner diameter of vacuum tubes in the first tubing set and the second tubing set can be about 0.110 inches. The first tubing set 1302 can also include an electrical channel 1304. The electrical channel includes multiple electrical leads to communicate electrical signals between the mounts 4400a-c and the cassette 1200, and can be coupled to the electrical interface. Sensors and electrical components in the cassette 1200 can also be coupled to the electrical interface.

The second tubing set 1316 includes one or more tube groups 1318a-c, each tube subgroup configured to provide a mount 4400a-c with saline, contrast, and vacuum. Each tube group 1318a-c can also provide each mount 4400a-c with one or more electrical leads. In the example in FIG. 21, each tube group 1318a-c includes a proximal end 1320 coupled to the splitter 1310 and a distal end 1322 coupled to one of the one or more mounts 4400a-c. Each tube group 1318a-c includes a saline subchannel in communication with the second saline flow-path in the splitter 1310, a contrast subchannel in communication with the second contrast flow-path in the splitter 1310, and a vacuum subchannel in communication with the second vacuum flow-path in the splitter 1310. Each tube group 1318a-c can be configured in a ribbon-like design. In some embodiments, the length 1326 of the second tubing set can be between about 1 foot and about 4 feet. In a specific embodiment, the length 1326 of the second tubing set is about 2 feet. In this embodiment, the length of tubing in the first tubing set and the second tubing set needed to be inspected is significantly reduced from 42 feet to 18 feet, which correspondingly drastically reduces the inspection time to about 3-4.5 minutes, advantageously reduces set-up time such that the medical procedure (e.g., a thrombectomy) can begin much sooner, which increases the patient's likelihood of a more successful recovery. In addition, this significant reduction in the amount of tubing to inspect also minimizes glove changes by the medical practitioner inspecting the tubing set from three glove changes to one glove change. The second tubing set 1316 can also provide one or more electrical leads 1328 to another mount that does not require fluids, for example, a guide wire mount 4400d configured to axially move and rotate a guide wire 4405 coupled to the guide wire mount 4400d, the guide wire 4405 configured to be positioned partially or fully in the lumen of access catheter 4402c. In some embodiments, connectors may be used to couple the second tubing set 1316 to the cassette 1200 and/or the splitter 1310. However, use of connectors in fluidic systems can cause air bubbles to be formed and/or trapped in a portion of the connector. In this embodiment, advantageously connectors are not used, rather the second tubing set 1316 is coupled to the splitter 1310 and the mounts 4400a-d such that the second tubing set 1316 is not meant to be decoupled prior to or during a medical procedure (for example, permanently, semi-permanently).

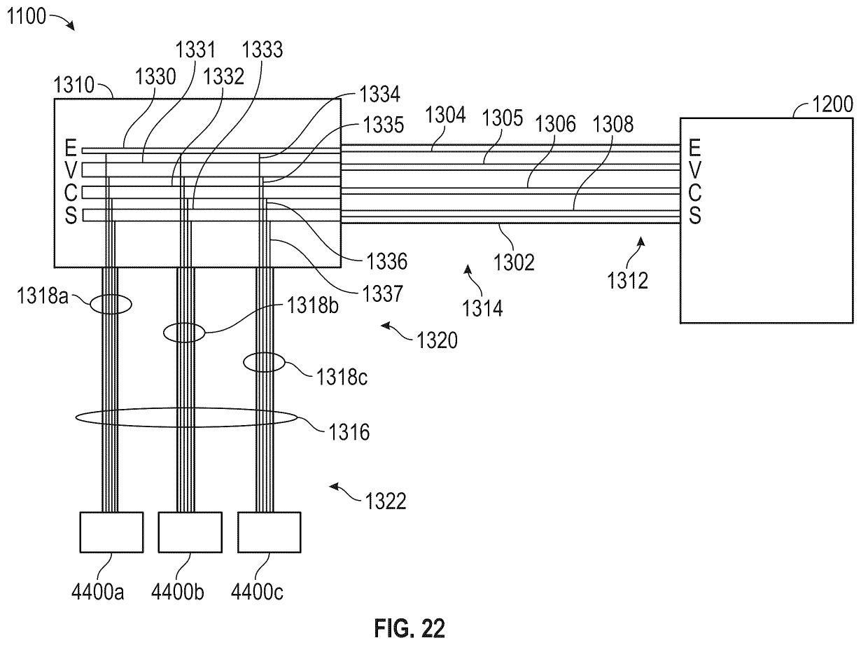

FIG. 22 is a schematic of an example of the fluidics assembly 1100 illustrated in FIG. 21 illustrating some additional features. The fluidics assembly 1100 includes cassette 1200, splitter 1310, and one or more mounts 4400a-c, first tubing set 1302 coupled to the cassette 1200 and splitter 1310, and second tubing set 1316 coupled between the splitter 1310 and the one or more mounts 4400a-c, the first tubing set 1302 having a saline channel 1308, a contrast channel 1306, and a vacuum channel 1305 for providing saline, contrast, and vacuum to the splitter 1310. In this example, the fluidics assembly 1100 is shown having three mounts 4400a-c that receive saline, contrast, and vacuum from the cassette 1200. The first tubing set 1302 also includes an electrical channel 1304 having a plurality of electrical leads for connecting electrical components in the mounts 4400a-c to an electrical interface of the cassette 1200. The second tubing set 1316 includes tube groups 1318a-c providing saline, contrast, and vacuum to mounts 4400a-c. Each of the tube groups 1318a-c includes a saline subchannel 1337, a contrast subchannel 1336, a vacuum subchannel 1335, and an electrical subchannel 1334.

The splitter 1310 is structured to provide fluid communication of saline from the saline channel 1308 to the multiple saline subchannels 1337 in the tube groups, to provide fluid communication of contrast from the contrast channel 1306 to the multiple contrast subchannels 1336 in the tube groups, to provide fluid communication of saline from the single saline channel 1308 to the multiple saline subchannels 1337 in the tube groups, and to provide fluid communication of vacuum from the vacuum channel 1305 to the multiple vacuum subchannels 1335 in the tube groups. Also, the splitter 1310 is structured to provide electrical connection between the electrical channel 1304 and the electrical subchannels 1334 in the tube groups. As illustrated in FIG. 22, the splitter 1310 includes a saline manifold 1333 coupled to the saline channel 1308 and the saline subchannels 1337 of each tube group 1318a-c in the second tubing set 1316, a contrast manifold 1332 coupled to the contrast channel 1306 and the contrast subchannels 1336 of each tube group 1318a-c in the second tubing set 1316, a vacuum manifold 1331 coupled to the vacuum channel 1305 and the vacuum subchannel of each tube group 1318a-c in the second tubing set 1316, and an electrical bus 1330 coupled to the electrical subchannels 1334 of each tube group 1318a-c in the second tubing set 1316. The saline manifold 1333, contrast manifold 1332, and vacuum manifold 1331 can be structured in various configurations that include tubes, splitters, manifolds, or any other structures to provide fluid communication from a single channel in the first tubing set 1302 to multiple subchannels in the second tubing set 1316. The electrical bus 1330 can be any electrical structure that provides electrical communication between the electrical subchannels of the tube groups 1318a-c in the second tubing set 1316 and the electrical channel 1304 in the first tubing set, including separate electrical wires (leads) or other electrical connections. For example, the electrical subchannels in the tube groups can include a plurality of electrical leads which are bundled together in the splitter 1310 and form the electrical channel 1304 in the first tubing set. As shown in FIG. 21, the electrical bus 1330 can also electrically connect the electrical channel 1304 of the first tubing set 1302 to the one or more electrical leads 1328 to guide wire mount 4400d. Other embodiments having more or fewer mounts and corresponding connections are also possible. In some embodiments, the splitter 1310 may be referred to as a relay cassette. In some embodiments, the splitter 1310 may be located outside of the sterile field or within the sterile field. In some embodiments, the splitter 1310, second tubing set 1316, and associated hub assemblies (e.g., mounts 4400a, 4400b, and 4400c and associated hubs) can be positioned within the sterile field.

The embodiment of communication channels 1300 illustrated in FIGS. 21 and 26 is designed to minimize the time needed for inspecting the fluid lines. In this example, the fluid communication channels 1300 include a first tubing set 1302 having a proximal end 1312 coupled to the cassette 1200 and a distal end 1314 coupled to a splitter 1310. The fluid communication channels also include a second tubing set 1316 having one or more tube groups 1318a-c, each tube group 1318a-c having a proximal end 1320 coupled to the splitter 1310 and a distal end 1322 coupled to one of the mounts 4400a-c. However, use of connectors in fluidic systems can cause air bubbles to be formed and/or trapped in a portion of the connector. In some preferred embodiments, advantageously connectors (i.e., releasable connectors) are not used to couple the first tubing set 1302 to the cassette 1200 and the splitter 1310, and are not used to connect the second tubing set 1316 to the splitter 1310. Instead, the first tubing set 1302 is coupled to the cassette 1200 and the splitter 1310 in a persistent coupling (e.g., permanent connection, semi-permanent coupling using glue, clamped fittings, integrally molded portions, and/or other suitable couplings) that it is not meant to be decoupled prior to or during a medical procedure. In preferred embodiments, the first tubing set 1302 and the second tubing set 1316 includes transparent tubing. The cassette 1200 can include fluid communication channels (including portions of components) configured to communicate saline, contrast, and vacuum in first saline, contrast, and vacuum flow-paths, respectively. The splitter 1310 can include fluid communication channels configured to communicate saline, contrast, and vacuum in the second saline, contrast, and vacuum flow-paths, respectively. For example, as described in reference to FIG. 22. The fluid communication channels in the cassette 1200 and the splitter 1310 can include one or more tubes, channels (e.g., molded channels), portions of components (e.g., a drip chamber, clot pod, at least a portion of peristaltic saline pump, at least a portion of a contrast pump, robotically controlled valves, etc.) and other suitable fluid communication structures. The proximal end of the first tubing set can be coupled to the cassette 1200 using a persistent coupling. In some embodiments, the proximal end of the first tubing set 1302 extends into the cassette 1200 and forms a portion of a channel in the cassette 1200. The distal end of the first tubing set can be coupled to the splitter 1310 using a persistent coupling. In some embodiments, the distal end of the first tubing set 1302 extend into the splitter 1310 and can form a portion of a channel in the splitter 1310. In some embodiments, a portion of, or all of, the fluid communication channels of the first tubing set 1302 are integrally formed with a portion of, or all of, and the fluid communication channels of the splitter 1310. In an example, tubes forming each of a saline channel 1308, a contrast channel 1306, and a vacuum channel 1305 of the first tubing set 1302 are integrally formed with tubes forming saline, contrast, and vacuum fluid communication channels of the splitter 1310. In some embodiments, a portion of, or all of, the fluid communication channels of the second tubing set 1316 are integrally formed with a portion of, or all of, and the fluid communication channels of the splitter 1310. In an example, a portion, or all of, tubes forming a saline subchannel, a contrast subchannel, and a vacuum subchannel in each of one or more tube groups 1318a-c are formed to be integral with structures (e.g., tubes) forming the fluid communication channels of the splitter (e.g., saline subchannel 1337, contrast subchannel 1336, vacuum subchannel 1335, manifolds 1333, 1332, 1331). In some embodiments, all or part of the fluid communication channels of the first tubing set 1302, the splitter 1310, and the second tubing set 1316 are formed as in integral structure and are persistently connected. In some embodiments, all or part of the fluid communication channels of the first tubing set 1302, the splitter 1310, the second tubing set 1316 and at least some of the fluid communication structures in the mounts 4400a-c are formed as an integral structure such that they are persistently connected. In some embodiments, all of the fluid communication channels in the first tubing set 1302, the splitter 1310, the second tubing set 1316 include tubing. For example, tubing that is coupled to the cassette 1200, split into multiple tube groups in the splitter, and tubing that is coupled to the splitter and the mounts. In some embodiments, all of the fluid communication channels in the first tubing set 1302, the splitter 1310, the second tubing set 1316 and the mounts 4400a-c include tubing. Some embodiments can include non-persistent coupling of fluid communication channels, for example, it is determined that such connections do not heighten the likelihood trapping air bubbles. In certain embodiments, tubes that are integrally formed with one another may be referred to as “tube sections” that together form a tube.

In some embodiments, a splitter, such as splitter 1310, can be part of or coupled to a drive table.

Additional details for the fluidics system are described in U.S. patent application Ser. No. 18/666,217, entitled FLUIDICS CONTROL SYSTEM FOR MULTI CATHETER STACK, filed May 16, 2024, the entirety of which is hereby incorporated by reference herein.

Various systems and methods are described herein primarily in the context of a neurovascular access or procedure. However, the inventors contemplate applicability of the disclosed catheters, systems, and methods to any of a wide variety of alternative applications, including within the coronary vascular or peripheral vascular systems as well as other hollow organs or tubular structures in the body.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A neurovascular catheter for delivery through an aorta and into an ostium, the catheter comprising:
a tubular body extending between a proximal end to a distal end,
wherein the tubular body has a length of about 140 cm to about 180 cm, a 5 Fr size, and an internal diameter of about 0.065″ to about 0.067″,
the tubular body including a proximal portion and a distal portion, wherein the distal portion includes a polymer layer, and wherein the proximal portion includes a hypotube, wherein the tubular body comprises a layer of braids extending at least partially along the proximal portion and at least partially along the distal portion.

2. The catheter of claim 1, wherein the layer of braid extends from the proximal end up to a predetermined distance from the distal end.

3. (canceled)

4. The catheter of claim 2, wherein a distal most portion of the catheter within the predetermined distance from the distal end comprises a polymer with a lowest hardness value.

5. (canceled)

6. The catheter of claim 1, wherein the tubular body comprises a pre-shaped tip, the pre-shaped tip comprising one or more curves.

7. The catheter of claim 6, wherein the distal portion of the tubular body comprises a segment with Tungsten additives, the segment extending over the pre-shaped tip.

8. The catheter of claim 6, wherein a distal portion of the layer of braids is annealed.

9. (canceled)

10. (canceled)

11. The catheter of claim 8, wherein the distal portion that is annealed is shorter than a length of the pre-shaped tip.

12. (canceled)

13. (canceled)

14. The catheter of claim 1, wherein the lair of braids is located radially inward of the polymer layer.

15. The catheter of claim 1, wherein the layer of braids is located radially outward of the hypotube.

16. The catheter of claim 1, wherein the hypotube is made of Nitinol.

17. The catheter of claim 16, wherein the hypotube include a laser cut pattern.

18. The catheter of claim 17, wherein the laser cut pattern is a flexible laser cut pattern on proximal and distal ends of the hypotube.

19. The catheter of claim 18, wherein the hypotube is less stiff in proximal and distal ends with the flexible laser cut pattern.

20. The catheter of claim 19, wherein the flexible laser cut pattern comprises uninterrupted spiral cuts.

21. (canceled)

22. The catheter of claim 16, wherein the hypotube is configured to be packaged in a bent configuration and is further configured to resume a straight configuration upon being unpackaged.

23. (canceled)

24. (canceled)

25. (canceled)

26. The catheter of claim 1, wherein the distal portion of the tubular body is about 35 cm.

27. The catheter of claim 1, wherein the tubular body further comprises an inner lining layer extending from the proximal end to the distal end of the tubular body.

28. The catheter of claim 1, wherein the length of the tubular body is at least 175 cm, wherein the catheter is configured to be driven by a robotic system.

29. (canceled)

30. (canceled)

31. The catheter of claim 1, wherein a stiffness of the hypotube is greater than a stiffness of the polymer layer.

32. The catheter of claim 1, wherein the stiffness of the polymer layer increases from the distal end to the proximal end.

33.-38. (canceled)
